(19) United States
(12) Patent Application Publication
(10) Pub. No.: US 2013/0261372 A1
(43) Pub. Date: Oct. 3, 2013

(54) Device, System, and Method for Delivery of Sugar Glass Stabilized Compositions

(71) Applicants: Roderick A. Hyde, Redmond, WA (US); Jordin T. Kare, Seattle, WA (US); Gary L. McKnight, Bothell, WA (US); Nathan P. Myhrvold, Medina, WA (US); Tony Pan, Cambridge, MA (US); Elizabeth A. Sweeney, Seattle, WA (US); Lowell L. Wood, JR., Bellevue, WA (US)

(72) Inventors: Roderick A. Hyde, Redmond, WA (US); Jordin T. Kare, Seattle, WA (US); Gary L. McKnight, Bothell, WA (US); Nathan P. Myhrvold, Medina, WA (US); Tony Pan, Cambridge, MA (US); Elizabeth A. Sweeney, Seattle, WA (US); Lowell L. Wood, JR., Bellevue, WA (US)

(73) Assignee: Elwha LLC, a limited liability company of the State of Delaware

(21) Appl. No.: 13/435,511

(22) Filed: Mar. 30, 2012

(51) Int. Cl. A61M 37/00; A61M 31/00; A61M 5/168

(52) U.S. Cl. 600/9; 604/93.01; 604/285; 604/65; 604/22; 604/20

(57) ABSTRACT

Devices, methods, and compositions are described that includes an implantable device including one or more compartments. One or more pharmaceutically effective compounds stabilized in a sugar glass composition, at least one of the one or more stabilized pharmaceutically effective compounds in the sugar glass composition enclosed within the one or more compartments; and one or more reservoirs configured to provide access for one or more release agents to an interior of the sugar glass composition, wherein the one or more reservoirs are configured to controllably dispense the one or more release agents to disrupt the sugar glass composition from the interior of the sugar glass composition.

### SUMMARY

A drug delivery device is described herein that includes an implantable delivery device, oral delivery device, or rectal delivery device including one or more compartments and one or more pharmaceutically effective compounds stabilized in a sugar glass composition, at least one of the one or more stabilized pharmaceutically effective compounds in the sugar glass composition enclosed within the one or more compartments. The drug delivery device includes one or more reservoirs configured to provide access for one or more release agents to an interior of the sugar glass composition. The one or more reservoirs are configured to controllably dispense the one or more release agents to disrupt the sugar glass composition from an interior of the sugar glass composition. The one or more reservoirs can include one or more conduits or channels to provide access to the release agent within one or more containment vessels. The one or more reservoirs can include one or more receptacles for the release agent. The one or more reservoirs can include one or more conduits or channels to provide access to physiological fluids outside the drug delivery device. The physiological fluids can include, but are not limited to, gastric fluid, saliva, intestinal fluid, blood fluid, interstitial fluid, cerebrospinal fluid, or lymph fluid. The one or more release agents can be configured to disrupt the sugar glass composition from the interior to an exterior of the sugar glass composition.

The drug delivery device can include a controller configured to activate the one or more reservoirs to controllably dispense the one or more release agents to disrupt the sugar glass composition and to initiate release of the one or more therapeutic compounds from the sugar glass composition. The drug delivery device can include an energy transducer configured to degrade one or more membranes or covers on the one or more reservoirs to initiate release of the one or more release agents into the interior of the sugar glass composition.

The drug delivery device can be an implantable delivery device. The drug delivery device drug delivery device can include an orally deliverable or rectally deliverable device. The one or more reservoirs can be at least partially embedded in the sugar glass composition. The one or more reservoirs can include a channel to the interior of the sugar glass composition. The one or more reservoirs can be completely embedded within the sugar glass composition.

The device can include one or more containment vessels distal to the interior of the sugar glass composition, the one or more containment vessels configured to contain the one or more release agents and configured to deliver the one or more release agents through the one or more at least partially embedded reservoirs to the interior of the sugar glass composition. The device can include one or more containment vessels at the interior of the sugar glass composition, the one or more containment vessels configured to contain the one or more release agents and configured to deliver the one or more release agents through the one or more at least partially embedded reservoirs toward a region proximal to the interior of the sugar glass composition.

The one or more reservoirs can include one or more physical channels having a dispensing end proximal to the interior of the sugar glass composition, wherein the one or more release agents are configured to pass through the one or more physical channels and through the dispensing end to disrupt the sugar glass composition at the interior of the sugar glass composition.

The one or more reservoirs can include one or more conductive components having a dispensing end proximal to the interior of the sugar glass composition, wherein the one or more release agents are configured to pass through the one or more conductive components and controllably dispense through the dispensing end to disrupt the sugar glass composition at the interior of the sugar glass composition. The one or more conductive components can include one or more hydrophilic fibers configured to initiate hydration by controllably dispensing the one or more release agents through the dispensing end to the interior of the sugar glass composition. The one or more conductive components can include one or more microchannels or nanochannels configured to initiate hydration by controllably dispensing the one or more release agents through the dispensing end to the interior of the sugar glass composition. The one or more reservoirs can include one or more microparticles or microvesicles configured to initiate hydration by controllably dispensing the one or more release agents through the dispensing end to the interior of the sugar glass composition. The one or more release agents can include, but are not limited to, an aqueous solution, a physiologic solution, an ionic solution, a non-physiologic pH solution, an enzymatic agent, a degradative agent, or a biochemical agent.

The controller can be configured to activate the one or more reservoirs to controllably dispense the one or more release agents in response to one or more exogenous components. The one or more exogenous components can include a biochemical agent indicative of an environmental condition. The one or more exogenous components can include a pathogenic agent or an environmental agent. The one or more reservoirs can include one or more encapsulation matrices embedded in the sugar glass composition. The one or more reservoirs can include one or more controlled release polymers embedded in the sugar glass composition. The one or more reservoirs can include one or more covers configured to be activated by the one or more controllers. The controller can be configured to activate the one or more reservoirs to controllably dispense the one or more release agents in response to one or more endogenous components. The one or more endogenous components can be indicative of a disease or condition in a vertebrate subject. The one or more endogenous components can include, but are not limited to, physiologic fluid, physiologic pH, physiologic analytes, e.g., proteins, enzymes, ions, or salts, or biomarkers in a vertebrate subject. The biomarkers include any biological analyte indicative of a condition in the vertebrate subject, e.g., proteins, nucleic acids, antibodies, or cytokines. The one or more endogenous components can include a biochemical agent present in the vertebrate subject and indicative of normal biological processes, pathogenic processes, or pharmacologic responses to a therapeutic intervention, or indicative of a disease or condition in a vertebrate subject. The one or more reservoirs can include one or more controlled release polymers. The one or more controlled release polymers can include one or more hydrogels. The one or more reservoirs can include one or more covers configured to be activated by the one or more controllers.

The one or more release agents can include one or more endogenous components present in a vertebrate subject to disrupt the sugar glass composition from the interior of the sugar glass composition. The one or more endogenous components can include one or more physiological fluids, e.g., blood, lymph, gastric, saliva, or intestinal fluids. The one or more reservoirs can include one or more encapsulation matrices including one or more encapsulated release agents. The one or more encapsulation matrices can include pressurized microcapsules in the sugar glass composition.

The pressurized microcapsules can be configured to release the one or more release agents from the pressurized microcapsules into the sugar glass composition in a time dependent manner. The pressurized microcapsules can be configured to release the one or more release agents into the sugar glass composition responsive to acoustic energy. The device can include an energy transducer configured to initiate release of the one or more encapsulated release agents from the pressurized microcapsules into the sugar glass composition in the time dependent manner. The one or more encapsulation matrices can include one or more tuned microcapsules in the sugar glass composition. The one or more tuned microcapsules can be responsive to two or more different tunings. The device can include an energy transducer configured to initiate release of the one or more encapsulated release agents from the one or more tuned microcapsules into the sugar glass composition. The one or more tuned microcapsules can be configured to release the one or more release agents into the sugar glass composition responsive to acoustic energy. The energy transducer can be configured to initiate release of the one or more encapsulated release agents from the one or more tuned microcapsules into the sugar glass composition in a time dependent manner. The energy transducer can be an ultrasonic energy transducer.

The energy transducer can include, but is not limited to, an acoustic energy transducer, ultrasonic energy transducer, magnetic energy transducer, or electrical energy transducer. The energy transducer can be configured to be internal or external to the device. The one or more reservoirs can be configured to be activated to release the one or more release agents by at least one of pressure variation, temperature variation, or variation in wavelength exposure to radiation. The pharmaceutically effective compound can include a therapeutic compound or a prophylactic compound. The pharmaceutically effective compound can include, but is not limited to, at least one of a vaccine, an adjuvant, a small molecule, or a biological agent. The biological agent can be, for example, a nucleic acid for a gene therapy agent. The sugar glass composition can include, but is not limited to, at least one of a monosaccharide, a disaccharide, a polysaccharide, or an oligosaccharide. The sugar glass composition can include, but is not limited to, at least one of trehalose glass, glucose glass, sugar glass. The sugar glass composition can include, but is not limited to, at least one of dextran, phosphatidylcholine, hexuronic acid, polyethylene glycol, or sugar alcohol. The sugar glass composition can include, but is not limited to, an amino acid, metal, plastic, or salt, e.g., metal carboxylate glass, borosilicate glass, acrylic glass, aluminum oxynitride glass, Muscovite glass, or calcium phosphate glass.

A method is described herein that includes enclosing one or more pharmaceutically effective compounds stabilized in a sugar glass composition within one or more compartments of an implantable device; and enclosing the one or more release agents in one or more reservoirs, wherein the one or more reservoirs are configured to provide access for one or more release agents to an interior of the sugar glass composition, and wherein the one or more reservoirs are configured to controllably dispense the one or more release agents to disrupt the sugar glass composition from the interior of the sugar glass composition. The one or more reservoirs can be configured to controllably release the one or more pharmaceutically effective compounds from the sugar glass composition.

In the method, enclosing the one or more release agents in the one or more reservoirs can include enclosing the one or more release agents in one or more physical channels having a dispensing end proximal to the interior of the sugar glass composition, wherein the one or more release agents are configured to pass through the one or more physical channels and through the dispensing end to penetrate the sugar glass composition at the interior of the sugar glass composition. In the method, enclosing the one or more release agents in the one or more reservoirs can include enclosing the one or more release agents in one or more conductive components having a dispensing end proximal to the interior of the sugar glass composition, wherein the one or more release agents are configured to pass through the one or more conductive components and through the dispensing end to disrupt the sugar glass composition at the interior of the sugar glass composition.

The method can include at least partially embedding the one or more reservoirs in the sugar glass composition. The one or more reservoirs can include a channel to the interior of the sugar glass composition. The method can include completely embedding the one or more reservoirs within the sugar glass composition. The one or more conductive components can include one or more hydrophilic fibers configured to initiate hydration by controllably dispensing the one or more release agents through the dispensing end to the interior of the sugar glass composition. The one or more conductive components can include one or more microchannels or nanochannels configured to initiate hydration by controllably dispensing the one or more release agents through the dispensing end to the interior of the sugar glass composition. The one or more conductive components can include one or more microparticles or microvesicles configured to initiate hydration by controllably dispensing the one or more release agents through the dispensing end to the interior of the sugar glass composition.

The method can include encapsulating the one or more release agents within the one or more reservoirs to form one or more encapsulation matrices embedded in the sugar glass composition. The method can include encapsulating the one or more release agents within the one or more reservoirs to form one or more controlled release polymers embedded in the sugar glass composition. The method can include encapsulating the one or more release agents within the one or more encapsulation matrices to form pressurized microcapsules within the sugar glass composition. The method can include providing an energy transducer configured to initiate release of the one or more release agents from the pressurized microcapsules into the sugar glass composition. The method can include encapsulating the one or more release agents within the one or more encapsulation matrices to form tuned microcapsules in the sugar glass composition. The method can include providing an energy transducer configured to initiate release of the one or more release agents from the tuned microcapsules into the sugar glass composition. The method can include providing the energy transducer configured to initiate release of the one or more release agents from the tuned microcapsules into the sugar glass composition in a time dependent manner. The method can include releasing the one or more release agents from two or more differently tuned microcapsules.

A method for administering one or more pharmaceutically effective compounds to a subject is described herein that includes enclosing one or more pharmaceutically effective compounds stabilized in a sugar glass composition within one or more compartments of an implantable device; and enclosing the one or more release agents in one or more reservoirs, wherein the one or more reservoirs are configured to provide access for one or more release agents to an interior of the sugar glass composition, and wherein the one or more reservoirs are configured to controllably dispense the one or more release agents to disrupt the sugar glass composition from the interior of the sugar glass composition. The method can include controllably releasing the one or more pharmaceutically effective compounds from the sugar glass composition into the subject.

In the method, enclosing the one or more release agents in the one or more reservoirs can include enclosing the one or more release agents in one or more physical channels having a dispensing end proximal to the interior of the sugar glass composition, wherein the one or more release agents are configured to pass through the one or more physical channels and through the dispensing end to penetrate the sugar glass composition at the interior of the sugar glass composition. In the method, enclosing the one or more release agents in the one or more reservoirs can include enclosing the one or more release agents in one or more conductive components having a dispensing end proximal to the interior of the sugar glass composition, wherein the one or more release agents are configured to pass through the one or more conductive components and through the dispensing end to disrupt the sugar glass composition at the interior of the sugar glass composition. The method can include at least partially embedding the one or more reservoirs in the sugar glass composition. The one or more reservoirs can include a channel to the interior of the sugar glass composition. The method can include completely embedding the one or more reservoirs within the sugar glass composition.

A system comprising is described herein that includes a drug delivery device including one or more compartments; one or more pharmaceutically effective compounds stabilized in a sugar glass composition, at least one of the one or more stabilized pharmaceutically effective compounds in the sugar glass composition enclosed within the one or more compartments; and one or more reservoirs configured to provide access for one or more release agents to an interior of the sugar glass composition, wherein the one or more reservoirs are configured to controllably dispense the one or more release agents to disrupt the sugar glass composition from the interior of the sugar glass composition. The drug delivery device can be an implantable delivery device. The drug delivery device can include an orally deliverable or rectally deliverable device.

The system can include a drug delivery device including a controller configured to activate the one or more reservoirs to controllably dispense the one or more release agents to disrupt the sugar glass composition and to initiate release of the one or more therapeutic compounds from the sugar glass composition. The system can include can include a drug delivery device including an energy transducer configured to degrade one or more membranes or covers on the one or more reservoirs to initiate release of the one or more release agents into the interior of the sugar glass composition.

The one or more reservoirs can be at least partially embedded in the sugar glass composition. The one or more reservoirs can include a channel to the interior of the sugar glass composition. The one or more reservoirs can be completely embedded within the sugar glass composition. The one or more reservoirs can include one or more conduits or channels to provide access to the release agent within one or more containment vessels. The one or more reservoirs can include one or more receptacles for the release agent. The one or more reservoirs can include one or more conduits or channels to provide access to physiological fluids outside the drug delivery device. The one or more release agents can be configured to disrupt the sugar glass composition from the interior to an exterior of the sugar glass composition.

The one or more reservoirs can include one or more physical channels having a dispensing end proximal to the interior of the sugar glass composition, wherein the one or more release agents are configured to pass through the one or more physical channels and through the dispensing end to disrupt the sugar glass composition at the interior of the sugar glass composition. The one or more reservoirs can include one or more conductive components having a dispensing end proximal to the interior of the sugar glass composition, wherein the one or more release agents are configured to pass through the one or more conductive components and controllably dispense through the dispensing end to disrupt the sugar glass composition at the interior of the sugar glass composition. The one or more reservoirs can include one or more microparticles or microvesicles configured to initiate hydration by controllably dispensing the one or more release agents through the dispensing end to the interior of the sugar glass composition. The system can include a drug delivery device including one or more containment vessels distal to the interior of the sugar glass composition, the one or more containment vessels configured to contain the one or more release agents and configured to deliver the one or more release agents through the one or more at least partially embedded reservoirs to the interior of the sugar glass composition. The system can include a drug delivery device including one or more containment vessels at the interior of the sugar glass composition, the one or more containment vessels configured to contain the one or more release agents and configured to deliver the one or more release agents through the one or more at least partially embedded reservoirs toward a region proximal to the interior of the sugar glass composition. A composition is described herein that includes one or more pharmaceutically effective compounds stabilized in a sugar glass composition; and one or more release agents enclosed in one or more reservoirs, wherein the one or more reservoirs are at least partially embedded within an interior of the sugar glass composition. The one or more reservoirs can be embedded within the sugar glass composition. The one or more pharmaceutically effective compounds can comprise one or more therapeutic compounds. The one or more pharmaceutically effective compounds can comprise one or more prophylactic compounds. The one or more reservoirs can comprise a channel to the interior of the sugar glass composition. The one or more release agents can comprise an aqueous solution, a physiologic solution, an ionic solution, a non-physiologic pH solution, an enzymatic agent, a degradative agent, or a biochemical agent. The one or more reservoirs can comprise one or more conduits or channels to provide access to the release agent within one or more containment vessels. The one or more reservoirs can comprise one or more receptacles for the release agent. The one or more reservoirs can comprise one or more conduits or channels to provide access to physiological fluids outside the drug delivery device.

The sugar glass composition can include, but is not limited to, at least one of a monosaccharide, a disaccharide, a polysaccharide, or an oligosaccharide. The sugar glass composition can include, but is not limited to, at least one of trehalose, sucrose, glucose, fructose, maltulose, iso-maltose, nigerose, cellubiulose, turanose, panose, isomaltotriose, stachyose, nystose, maltotetrose, maltopentose, maltohexose, maltopheptose, ubombo sugar, raffinose, arabinose, galactose, xylose, melibiose, salicin, esculin, arbutin, glycerol, arabinose, adonitol, sorbose, thamnose, dulcitol, melezitose, starch, glycogen, gentiobiose, lyxose, tagatose, fucose, arabitol, gluconate, inulin, dextran, erythritol, xylitol, maltose, lactose, dextrose, palatnitol, glucopyranosyl, glucopyranosyl, inositol, mannitol, lactitol, malto-dextran, or sorbitol. The sugar glass composition comprises polyvinylpyrrolidone, polyethylene glycol, hexuronic acid, or phosphatidylcholine. The sugar glass composition can include, but is not limited to, at least one of carboxylate, phosphate, nitrate, sulfate, or bisulfate.

The one or more pharmaceutically effective compounds can include, but is not limited to, a vaccine, adjuvant, small molecule, or biological agent. The one or more pharmaceutically effective compounds can include, but is not limited to, an organic or inorganic small molecule, clathrate or caged compound, protocell, coacervate, microcapsule, proteinoid, liposome, vesicle, small unilamellar vesicle, large unilamellar vesicle, large multilamellar vesicle, multivesicular vesicle, lipid layer, lipid bilayer, micelle, organelle, cell, membrane, nucleic acid, peptide, polypeptide, protein, glycopeptide, glycolipid, glycoprotein, sphingolipid, glycosphingolipid, peptidoglycan, lipid, carbohydrate, metalloprotein, proteoglycan, chromosome, nucleus, nitric oxide, nitric oxide synthase, amino acid, micelle, polymer, co-polymer, or piloxymer. The one or more pharmaceutically effective compounds can include, but is not limited to, an anti-tumor agent, antimicrobial agent, anti-viral agent, analgesic, antiseptic, anesthetic, diagnostic agent, anti-inflammatory agent, vaccine, cell growth inhibitor, cell growth promoter, chemical debridement agent, immunogen, antigen, radioactive agent, apoptotic promoting factor, angiogenic factor, anti-angiogenic factor, hormone, enzymatic factor, enzyme, papain, collagenase, protease, peptidase, elastase, urea, vitamin, mineral, nutraceutical, cytokine, chemokine, probiotic, coagulant, anti-coagulant, phage, prodrug, prebiotic, blood sugar stabilizer, smooth muscle cell activator, epinephrine, adrenaline, neurotoxin, neuro-muscular toxin, Botulinum toxin type A, microbial cell or component thereof, or virus or component thereof. The composition can comprise at least one carrier fluid.

The one or more release agents can include, but is not limited to, at least one phase of water, saline, intravenous fluid, or other fluid. The one or more release agents can include, but is not limited to, at least one of an aqueous solution, buffered aqueous solution, physiologic solution, non-physiologic pH solution, ionic solution, enzymatic agent, degradative agent, or biochemical agent. The one or more release agents can include an exogenous agent, for example, provided in a containment reservoir. The one or more release agents can include an exogenous agent, for example, a pathogenic agent, an environmental agent, or a biochemical agent indicative of an environmental condition. The one or more release agents can include an endogenous agent, for example, a physiological fluid; gastric fluid such as an enzyme, acid, base, or other degradative agent; intestinal fluid; blood fluid, such as plasma; cerebrospinal fluid; or an interstitial fluid, any of which may be conducted fluidically, accumulated, or provided via a reservoir. The sugar glass composition can be soluble in the one or more release agents. The sugar glass composition can be immiscible in the one or more release agents. The composition can comprise at least one preservative. The at least one preservative can be at least one enzyme inhibitor. The at least one preservative can include, but is not limited to, at least one of validamycin A, TL-3, sodium orthovanadate, sodium fluoride, N-α-tosyl-Phe-chloromethylketone, N-α-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor. The at least one preservative can be a cryoprotectant. The composition can comprise at least one buffer. The at least one buffer can include, but is not limited to, at least one of bicarbonate, monosodium phosphate, disodium phosphate, or magnesium oxide. The sugar glass composition can be stable without refrigeration. The sugar glass composition can be in the form of at least one of particles, filaments, sheets, blocks, powder, or a mixture thereof. The sugar glass composition can comprise at least two layers. The at least two layers can include at least one sugar glass composition layer that is different from at least one other sugar glass composition layer. The at least two layers can include at least one pharmaceutically effective compound layer that is different from at least one other pharmaceutically effective compound layer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial diagrammatic view of an illustrative embodiment of a drug delivery device.

FIGS. 2A and 2B are partial diagrammatic views of an illustrative embodiment of a drug delivery device.

FIGS. 3A and 3B are partial diagrammatic views of an illustrative embodiment of a drug delivery device.

FIG. 4 is a partial diagrammatic view of an illustrative embodiment of a method.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

A drug delivery device is described herein that includes an implantable delivery device, oral delivery device, or rectal delivery device including one or more compartments and one or more pharmaceutically effective compounds stabilized in a sugar glass composition, at least one of the one or more stabilized pharmaceutically effective compounds in the sugar glass composition enclosed within the one or more compartments. The drug delivery device includes one or more reservoirs configured to provide access for one or more release agents to an interior of the sugar glass composition. The one or more reservoirs can include one or more of a containment vessel, a receptacle, or a conduit for fluids. The one or more reservoirs are configured to controllably dispense the one or more release agents to disrupt the sugar glass composition from an interior of the sugar glass composition. The one or more reservoirs can include one or more conduits or channels to provide access to the release agent within one or more containment vessels. The one or more reservoirs can include one or more receptacles for the release agent. The one or more reservoirs can include one or more conduits or channels to provide access to physiological fluids outside the drug delivery device. The one or more reservoirs can be at least partially embedded in the sugar glass composition. The one or more reservoirs can be completely embedded in the sugar glass composition. The physiological fluids can include, but are not limited to, gastric fluid, saliva, intestinal fluid, blood fluid, interstitial fluid, cerebrospinal fluid, or lymph fluid. The one or more release agents can be configured to disrupt the sugar glass composition from the interior to an exterior of the sugar glass composition.

A drug delivery device is described herein that includes an implantable delivery device, oral delivery device, or rectal delivery device including one or more compartments containing one or more pharmaceutically effective compounds. The drug delivery device includes the one or more pharmaceutically effective compounds that are stabilized in a sugar glass composition. The one or more compartments of the implantable delivery device, oral delivery device, or rectal delivery device can include a physical compartment that is at least partially covered with a thin metal membrane, a polymer membrane, or a hydrogel membrane. The one or more reservoirs can include one or more of a containment vessel, a receptacle, or a conduit for fluids. The one or more reservoirs can enclose one or more release agents within the sugar glass composition. The one or more reservoirs are configured to controllably dispense the one or more release agents to disrupt the sugar glass composition from an interior of the sugar glass composition. The one or more pharmaceutically effective compounds are stabilized in a sugar glass composition to resist changes in temperature and other degradation or destabilization factors during storage or shipment of the pharmaceutically effective compound. The one or more reservoirs can be at least partially embedded in the sugar glass composition. The one or more reservoirs can be completely embedded in the sugar glass composition.

In some aspects, the one or more compartments of the oral delivery device or rectal delivery device can include the sugar glass composition and one or more reservoirs enclosing one or more release agents within the sugar glass composition. In some aspects, the one or more compartments do not include a physical compartment to cover the sugar glass composition. The one or more reservoirs provide access for the one or more release agents to an interior of the sugar glass composition. Upon oral delivery or rectal delivery of the device, simultaneous access to an exterior and an interior of the sugar glass composition is provided for the release agents to disrupt the sugar glass composition from the exterior and the interior of the sugar glass composition.

The implantable delivery device, oral delivery device, or rectal delivery device including the one or more compartments having degradable thin metal membrane coverings or degradable polymer coverings over the sugar glass composition can include an energy transducer, e.g., a microchip with circuitry and a small battery to supply current to thermally disrupt the individual thin metal membrane coverings, or ultrasound to disrupt the degradable polymer covering. The individual compartment coverings may be disrupted automatically, e.g., programmed in the microchip, or by external command to execute a drug delivery schedule, meet dosing requirements and deliver multiple medications. Prior to removal of the individual thin metal membrane coverings, the one or more reservoirs embedded in the sugar glass composition are disrupted from an interior of the sugar glass composition to release the one or more release agents to dissolve the sugar glass composition. This is followed by disruption of the one or more thin metal membrane coverings to release the pharmaceutically effective composition from the compartment of the drug delivery device and deliver a dosage to a subject in need thereof. An energy transducer can include, but is not limited to, an acoustic energy transducer, ultrasonic energy transducer, magnetic energy transducer, or electrical energy transducer.

The drug delivery device including the stabilized pharmaceutically effective compound in a sugar glass composition can efficiently deliver one or multiple dosage forms of the pharmaceutically effective compound over varying dosage amounts and on a determined time schedule. The drug delivery device including the stabilized pharmaceutically effective compound can efficiently deliver multiple dosage forms and multiple dosage amounts, for example, one or more bolus administrations of the pharmaceutically effective compound at varying concentrations and/or times from the drug delivery device.

Stabilization of the one or more pharmaceutically effective compounds in the sugar glass composition can protect the pharmaceutically effective compounds from processing and storage-related stresses throughout the life of the compounds that can result in significant degradation and loss of bioactivity and can raise safety concerns. Various storage-related stresses on the pharmaceutically effective compounds can include elevated temperatures, exposure to liquid and solid hydrophobic interfaces, and vigorous mechanical agitation.

The one or more compartments of the drug delivery device can include the sugar glass composition, wherein the one or more pharmaceutically effective compounds are stabilized in the sugar glass composition. The one or more compartments of the device can include the one or more reservoirs at least partially embedded in the sugar glass composition. One or more release agents are enclosed in the one or more reservoirs within the sugar glass composition. The drug delivery device is configured to controllably dispense the one or more release agents from the one or more reservoirs to disrupt the sugar glass composition from an interior of the sugar glass composition. The drug delivery device can release a timed and measured dosage, e.g., a bolus dosage, of the pharmaceutically acceptable compound. The release agent within the reservoir can include one or more of an aqueous solution, buffered aqueous solution, physiologic solution, non-physiologic pH solution, ionic solution, enzymatic agent, degradative agent, or biochemical agent.

The drug delivery device can include a controller configured to activate the one or more reservoirs to controllably dispense the one or more release agents to disrupt the sugar glass composition and to initiate release of the one or more therapeutic compounds from the sugar glass composition. The controller can be configured to activate the one or more reservoirs to controllably dispense the one or more release agents in response to one or more exogenous components or one or more endogenous components. The one or more exogenous components, e.g., a biochemical agent, a pathogenic agent, or an environmental agent, can interact with the controller to initiate activation of the one or more reservoirs by the controller. The one or more endogenous components, e.g., physiologic fluid, physiologic pH, physiologic analytes, or biomarkers, can act directly on the reservoir to release the release agents, or can interact with the controller to initiate activation of the one or more reservoirs by the controller.

Controllably dispensing the one or more release agents from the one or more reservoirs to disrupt the sugar glass composition from an interior of the sugar glass composition provides for dissolution of the sugar glass composition from an interior location to an exterior of the sugar glass composition in the compartment. This provides for controlled dissolution or essentially instantaneous dissolution of the sugar glass composition to release an accurately determined dosage of the pharmaceutically acceptable compound from the compartment of the drug delivery device.

The one or more reservoirs at least partially embedded in the sugar glass composition contain the one or more release agents. The reservoirs containing release agent can include one or more of microchannels, nanochannels, microbubbles, hydrophilic fibers, or microencapsulation particles. The one or more sugar glass compositions can incorporate strands of micro-diameter material, e.g., hydrophilic fibers embedded within the sugar glass composition, in order to provide for a high surface area for rapid drying action and compact storage. The sugar glass composition itself can be coated on a substrate (e.g., sheet, fiber, particle) to form hydrophilic microchannels or nanochannels into an interior region of the sugar glass composition.

One or more pharmaceutically effective compounds in a sugar glass composition can provide thermostabilization of the compounds based on the ability of nonreducing disaccharides, such as trehalose and sucrose, to form the sugar glass composition: an infinitely viscous anhydrous liquid (functionally a solid) in which molecules are immobilized and no chemistry can occur. This phenomenon underlies the ability of anhydrobiotic organisms to survive desiccation. Because of this property, these nonreducing sugars can be used as cryopreservants and excipients in spray-dried or lyophilized formulations of the pharmaceutically effective compounds in biopharmaceutical products and vaccines. See e.g., Alcock et al., Sci. Trans'. Med. 2: 19ra12, 2010, and Giri, et al., Advanced Materials, 23(42): 4861-4867, Nov. 9, 2011, which are incorporated herein by reference. The one or more pharmaceutically effective compounds, e.g., one or more therapeutic compounds or one or more prophylactic compounds can be designed to maintain structure and functionality by stabilization of the compound in the sugar glass composition. The influence of freezing rate, buffer composition, and type of carbohydrate on the structure and activity of the therapeutic compound or the prophylactic compound, after freezing and freeze-drying, respectively, can be determined. Carbohydrates that can be used to form the sugar glass composition include, but are not limited to, disaccharide (trehalose), oligosaccharide (inulin) and polysaccharide (dextran). The therapeutic compound or the prophylactic compound can include, but is not limited to, a vaccine, a viral subunit vaccine, an attenuated live viral vaccine, a protein therapeutic, adjuvant, small molecule (peptide, protein, hormone, nucleic acid, antibody or antibody fragments, antigen-protein), or biological agent (bacteria, virus, eukaryotic or prokaryotic cell, liposome, phage). See e.g., Alcock et al., Sci. Transl. Med. 2: 19ra12, 2010, which is incorporated herein by reference.

The drug delivery device as described herein can be used for formulation and drug delivery of one or more pharmaceutically effective compounds, e.g., therapeutic compounds or prophylactic compounds, in a sugar glass composition. Improvement of vaccine formulations may be obtained by developing stable therapeutic or prophylactic compounds in the dry state in the sugar glass composition. Carbohydrates in the sugar glass compositions can be used to protect various types of drug compositions such as proteins and antigen/protein vaccines during freezing, drying and subsequent storage. When properly dried, a proteinaceous drug is incorporated in a matrix comprising the sugar glass composition in an amorphous glassy state. The stabilizing effect of the sugar glass composition may derive from the formation of a matrix which strongly reduces diffusion and molecular mobility (vitrification) and acts as a physical barrier between particles or molecules (particle/molecule isolation). Both the lack of mobility and the physical barrier provided by the matrix of the sugar glass composition can prevent aggregation and degradation of the dried therapeutic compound or prophylactic compound. Moreover, during the lyophilization process, the water molecules that form hydrogen bonds with the pharmaceutically effective compounds are replaced by the hydroxyl groups of the carbohydrate, by which the three dimensional structure/structural integrity of the pharmaceutically effective compound is maintained. See e.g., Amorij et al., Vaccine 25: 6447-6457, 2007 which is incorporated herein by reference.

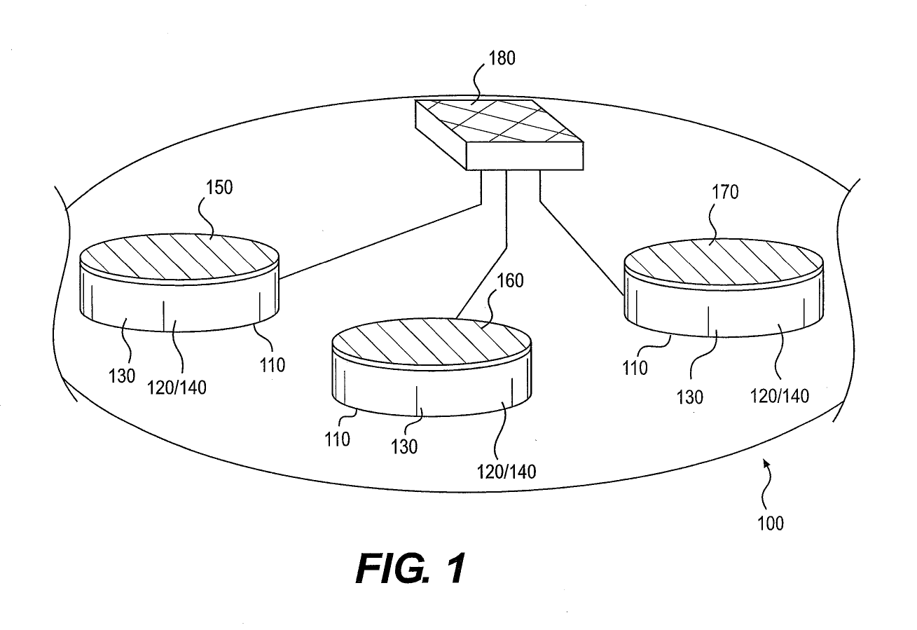

With reference to the figures, and with reference now to FIGS. 1 through 4, depicted is an aspect of a device, system, or method that can serve as an illustrative environment of and/or for subject matter technologies. The specific devices and methods described herein are intended as merely illustrative of their more general counterparts.

Referring to FIG. 1, depicted is a partial diagrammatic view of an illustrative embodiment of a drug delivery device 100 including an implantable delivery device, oral delivery device, or rectal delivery device including one or more compartments 110, one or more reservoirs 120 at least partially embedded in the sugar glass composition 130 containing a pharmaceutically effective composition, one or more reservoirs 120 configured to provide access for one or more release agents 140 to an interior of the sugar glass composition 130, wherein the one or more reservoirs 120 are configured to controllably dispense the one or more release agents 140 to disrupt the sugar glass composition 130 from the interior of the sugar glass composition. An external view of each of the one or more compartments 110 can include a metal membrane cover or polymer membrane cover 150 160 170. The metal membrane cover or the polymer membrane cover 150 160 170 can have an electrical connection to the metal membrane or a chemical connection to the polymer membrane via a controller 180 programmed to sequentially disrupt the individual membrane on each compartment 110 and expose the disrupted sugar glass composition 130 containing the pharmaceutically effective composition to the surrounding medium.

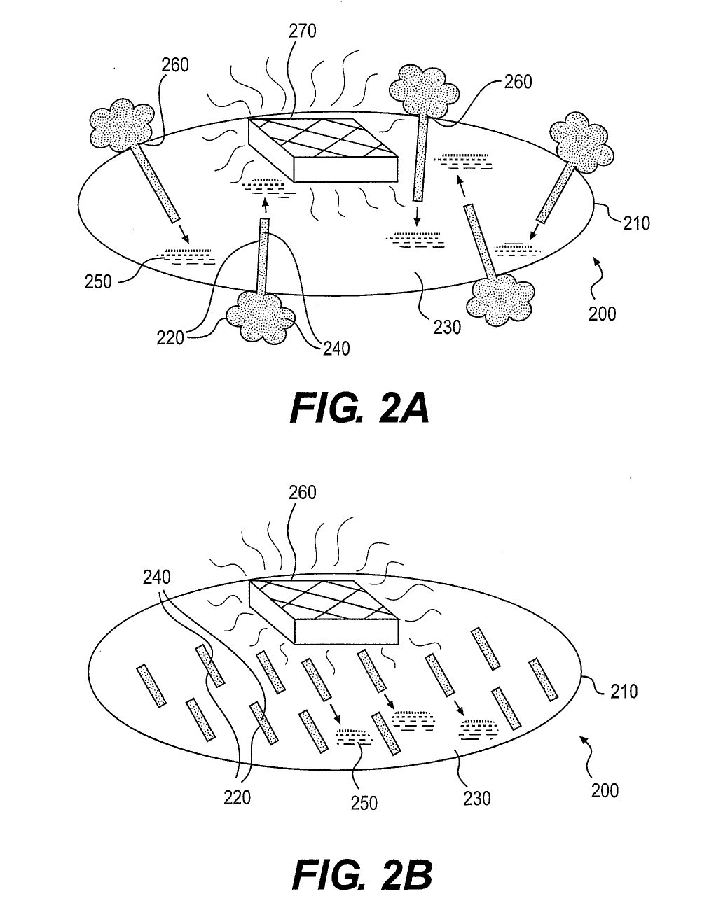

Referring to FIG. 2A, depicted is a partial diagrammatic view of an illustrative embodiment of a drug delivery device 200 including an implantable delivery device, oral delivery device, or rectal delivery device including one or more compartments 210, one or more pharmaceutically effective compounds stabilized in a sugar glass composition enclosed within the one or more compartments 210, and one or more reservoirs 220 configured to provide access for one or more release agents 240 to an interior of the sugar glass composition 230, wherein the one or more reservoirs 220 are configured to controllably dispense the one or more release agents 240 to disrupt or dissolve 250 the sugar glass composition 230 from the interior of the sugar glass composition. In an implantable delivery device, oral delivery device, or rectal delivery device, the one or more compartments 210 can include a physical container, as shown in FIG. 1, to contain the sugar glass composition including a pharmaceutically effective composition and one or more release agents 240 enclosed in one or more reservoirs 220. The one or more compartments 210/physical container can include a membrane, e.g., a thin metal membrane, a polymer membrane, or a hydrogel membrane, that can be removed to provide access to the sugar glass composition within the compartment. Alternatively, in an oral delivery device or rectal delivery device, the one or more compartments 210 can include the sugar glass composition without a physical container, the sugar glass composition including a pharmaceutically effective composition and one or more the one or more release agents 240 enclosed in one or more reservoirs 220 to disrupt or dissolve the sugar glass composition 230 from an interior of the sugar glass composition. The device can further include the one or more reservoirs 220 at least partially embedded in the sugar glass composition 230 containing a pharmaceutically effective composition. The one or more reservoirs 220 can be at least partially embedded in the sugar glass composition 230. For example, the one or more reservoirs 220 can be formed by etching on a silicon substrate. For example, the one or more reservoirs 220 can be formed by microchannels or nanochannels. The one or more reservoirs 220 can include a valve or membrane 260 wherein the valve or membrane 260 is disrupted to release the release agent from the reservoir. The valve or membrane can be located proximal 260 or distal 220 on the channel to an interior of the sugar glass composition 230. The drug delivery device 200 can include a controller 270 configured to activate the one or more reservoirs by opening the valve 260 or removing the membrane 260 to controllably dispense the one or more release agents to disrupt or dissolve the sugar glass composition and to initiate release of the one or more therapeutic compounds or prophylactic compounds from the sugar glass composition. The controller 270 can send an electrical, chemical, or ultrasonic signal from an energy transducer to disrupt the reservoir to release the release agents at an interior of the sugar glass composition. The energy transducer can be external to the delivery device or integral to the delivery device.

Referring to FIG. 2B, depicted is a partial diagrammatic view of an illustrative embodiment of a drug delivery device 200 including an implantable delivery device, oral delivery device, or rectal delivery device including one or more compartments 210, one or more pharmaceutically effective compounds stabilized in a sugar glass composition enclosed within the one or more compartments 210, and one or more reservoirs 220 configured to provide access for one or more release agents 240 to an interior of the sugar glass composition 230, wherein the one or more reservoirs 220 are configured to controllably dispense the one or more release agents 240 to disrupt or dissolve 250 the sugar glass composition 230 from the interior of the sugar glass composition. The device can further include the one or more reservoirs 220 at least partially embedded in the sugar glass composition 230 containing a pharmaceutically effective composition. The one or more reservoirs 220 can comprise a channel to the interior of the sugar glass composition 230. The one or more reservoirs may be completely embedded in the sugar glass composition. Alternatively, the one or more reservoirs 220 may be at least partially embedded in the sugar glass composition 230 and in contact with an outside surface of the compartment wherein the one or more reservoirs 220 dispense the one or more release agents 240 to disrupt or dissolve the sugar glass composition 230 from an interior of the sugar glass composition. The one or more reservoirs 220 can include one or more nanochannels or microchannels 220 containing the one or more release agents 240 and at least partially embedded in the sugar glass composition 230. The drug delivery device 200 can include a controller 260 configured to activate the one or more reservoirs 220 to controllably dispense the one or more release agents 240 to disrupt or dissolve 250 the sugar glass composition 230 and to initiate release of the one or more therapeutic compounds or prophylactic compounds from the sugar glass composition. The controller 260 can send an electrical, chemical, or ultrasonic signal from an energy transducer to disrupt the reservoir to release the release agents at an interior of the sugar glass composition. The energy transducer can be external to the delivery device or integral to the delivery device. Alternatively, the controller 260 can activate release of the one or more release agents 240, e.g., a physiological aqueous component, from the one or more reservoirs by allowing a physiological aqueous component to contact a hydrogel or polymer to dissolve the hydrogel or polymer and allow the aqueous component/release agent to pass through the one or more reservoirs to an interior of the sugar glass composition.

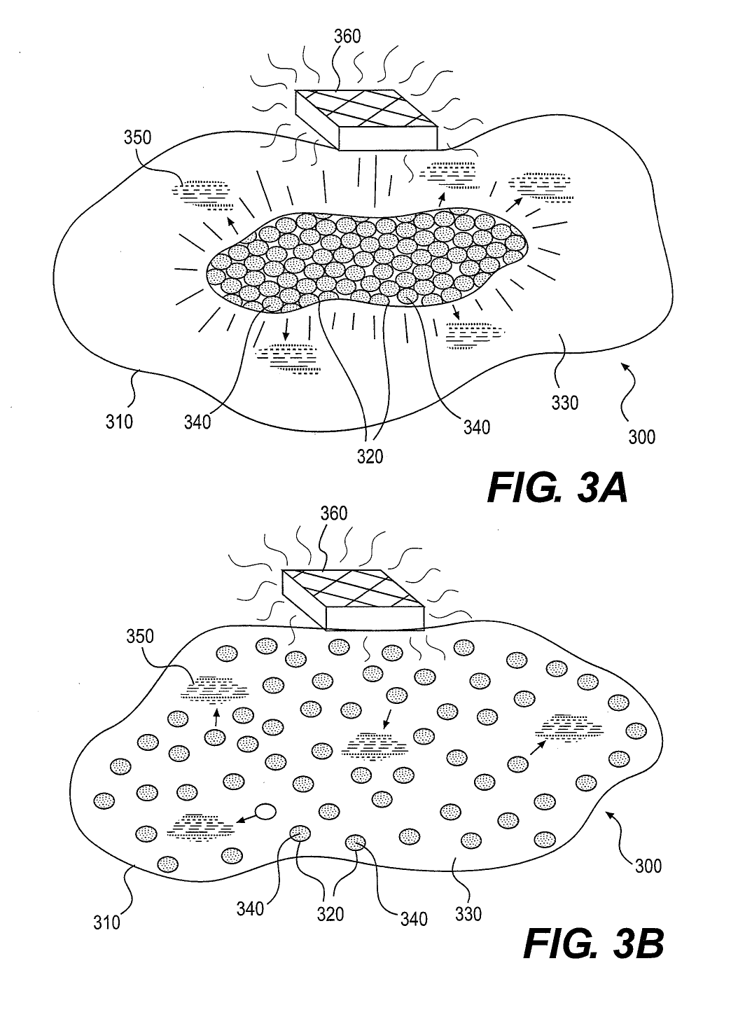

Referring to FIGS. 3A and 3B, depicted is a partial diagrammatic view of an illustrative embodiment of a drug delivery device 300 including an implantable delivery device, oral delivery device, or rectal delivery device including one or more compartments 310, one or more pharmaceutically effective compounds stabilized in a sugar glass composition enclosed within the one or more compartments 310, and one or more reservoirs 320 configured to provide access for one or more release agents 340 to an interior of the sugar glass composition 330, wherein the one or more reservoirs 320 are configured to controllably dispense the one or more release agents 340 to disrupt or dissolve 350 the sugar glass composition 330 from the interior of the sugar glass composition. The one or more reservoirs 320 can include one or more nanoparticles, microparticles, or microbubbles 320 containing the one or more release agents 340 and at least partially embedded in the sugar glass composition 330. In an implantable delivery device, oral delivery device, or rectal delivery device, the one or more compartments 310 can include a physical container, as shown in FIG. 1, to contain the sugar glass composition including a pharmaceutically effective composition and one or more release agents 340 enclosed in one or more reservoirs 320. The one or more compartments 310/physical container can include a membrane, e.g., a thin metal membrane, a polymer membrane, or a hydrogel membrane, that can be removed to provide access to the sugar glass composition within the compartment. Alternatively, in an oral delivery device or rectal delivery device, the one or more compartments 310 can include the sugar glass composition without a physical container, the sugar glass composition including a pharmaceutically effective composition and one or more the one or more release agents 340 enclosed in one or more reservoirs 320 to disrupt or dissolve the sugar glass composition 330 from an interior of the sugar glass composition. The drug delivery device 300 can include a controller 360 configured to activate the one or more reservoirs 320 to controllably dispense the one or more release agents 340 to disrupt or dissolve 350 the sugar glass composition 330 and to initiate release of the one or more therapeutic compounds or prophylactic compounds from the sugar glass composition. The controller 360 can send an electrical, chemical, or ultrasonic signal from an energy transducer to disrupt the reservoir to release the release agents at an interior of the sugar glass composition. The energy transducer can be external to the delivery device or integral to the delivery device.

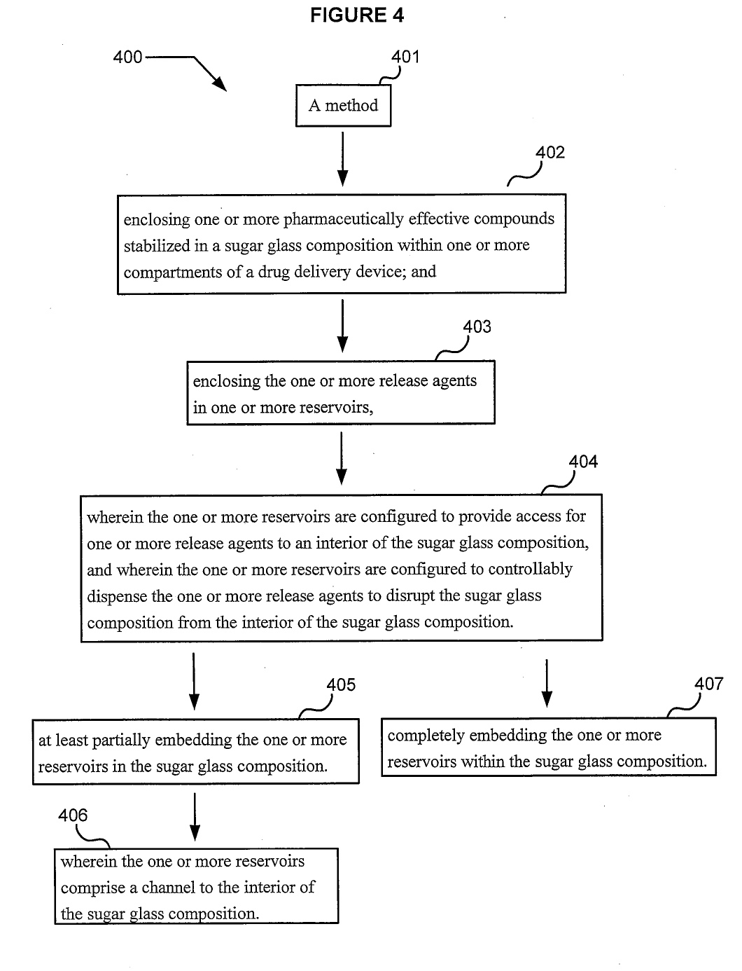

Referring to FIG. 4, depicted is a partial diagrammatic view of an illustrative embodiment of a method 401 that includes enclosing 402 one or more pharmaceutically effective compounds stabilized in a sugar glass composition within one or more compartments of a delivery device, e.g., an implantable delivery device, an oral delivery device, or a rectal delivery device; and enclosing 403 the one or more release agents in one or more reservoirs, wherein the one or more reservoirs 404 are configured to provide access for one or more release agents to an interior of the sugar glass composition, and wherein the one or more reservoirs are configured to controllably dispense the one or more release agents to disrupt the sugar glass composition from the interior of the sugar glass composition. The method can further include at least partially embedding 405 the one or more reservoirs in the sugar glass composition. The method can further include wherein the one or more reservoirs 406 comprise a channel to the interior of the sugar glass composition. The method can further include completely embedding 407 the one or more reservoirs within the sugar glass composition.

The one or more pharmaceutically effective composition, e.g., therapeutic compounds or prophylactic compounds, within a sugar glass composition can be loaded within a compartment of the device, frozen, and freeze-dried. The sugar glass composition includes reservoirs containing release agent, e.g., phosphate buffered saline contained in microcapsules, microspheres, microcylinders, microchannels, nanochannels, microbubbles, microparticles, or nanoparticles that have been embedded in the sugar glass composition. Nanoparticles containing a release agent can be prepared from a light-sensitive polymer formulated into nanoparticles that encapsulate the release agent. For example, the reservoir constructed of polymer nanoparticles undergoes self-destruction when irradiated with near infrared light at approximately 750 nm wavelength. Alternate polymers sensitive to different wavelengths of light can be used to construct polymer nanoparticles or coverings for one or more compartments containing different therapeutic compounds or prophylactic compounds.

The compartment can include a degradable coating over the sugar glass composition, such as a thin metal membrane covering, a polymer covering or a hydrogel covering, to separate individual compartments. The thin metal membrane coverings are fabricated using microchip fabrication methods that include sputtering and etching to create metal membranes of platinum and titanium. The compartment can include a degradable polymer covering over the sugar glass composition, for example, the light-sensitive copolymer of 4,5-dimethoxy-2-nitrobenzyl alcohol) and adipoyl chloride. The light-sensitive copolymer is a degradable coating that can be disrupted by a tuned laser at a specific wavelength. See, e.g., Fomina et al., J Am Chem. Soc. 132(28): 9540-9542, Jul. 21, 2010, which is incorporated herein by reference.

The drug delivery device can include the one or more compartments having degradable thin metal membrane coverings over the sugar glass composition and over the one or more reservoirs at least partially embedded in the sugar glass composition,

wherein the one or more reservoirs are configured to controllably dispense the one or more release agents to disrupt the sugar glass composition from an interior of the sugar glass composition. The drug delivery device can include a microchip with circuitry and a small battery to supply current (approximately 0.5 amp) to thermally disrupt the individual thin metal membrane coverings. The individual compartment coverings may be disrupted automatically, e.g., programmed in the microchip, or by external command to execute a drug delivery schedule, meet dosing requirements and deliver multiple medications.

The energy transducer can include, but is not limited to, an acoustic energy transducer, magnetic energy transducer, or electrical energy transducer. The energy transducer can include an ultrasonic energy transducer. The energy transducer can be integral to the drug delivery device or can be external to the drug delivery device. Upon removal of the individual thin metal membrane coverings, individual hydrogel coverings, or individual polymer coverings on the compartments or reservoirs, and/or disruption or dissolution of reservoirs that include microchannels, nanochannels, microbubbles, microparticles, or nanoparticles embedded in the sugar glass composition, the one or more release agents are delivered from the reservoir into the interior of the sugar glass composition where they act to disrupt or dissolve the sugar glass composition from dissolve the sugar glass composition.

The nanoparticle reservoirs comprised of light-sensitive copolymer and including one or more release agents can be disrupted by near infrared (NIR) irradiation that can penetrate up to 10 cm deep into tissues and be remotely applied with high spatial and temporal precision. The design of nanoparticle reservoirs comprised of light-sensitive copolymer relies on the photolysis of the multiple pendant 4-bromo7-hydroxycoumarin protecting groups to trigger a cascade of cyclization and rearrangement reactions leading to the degradation of the polymer backbone and release of the release agent from the nanoparticle reservoirs. The nanoparticle reservoirs comprised of polymeric material can disassemble in response to biologically benign levels of NIR irradiation upon two-photon absorption. See, e.g., Fomina et al., Macromolecules, 44: 8590-8597, September, 2011, which is incorporated herein by reference. The drug delivery device can be implanted into a subject, e.g., human, animal, or plant, and stored within the subject until needed. For example, the implant can be located just beneath the exterior of the subject, e.g., subcutaneously or subdermally. Alternatively, the drug delivery device can be designed and formulated for oral delivery or rectal delivery into the subject. The reservoirs and the degradable coating on the one or more compartments can be degraded upon one or more external or internal factor, e.g., external command or time interval.

In some instances, the one or more compartments on the implantable delivery device, oral delivery device, or rectal delivery device may include one or more coverings that comprise natural and/or synthetic stimulus-responsive hydrogel or polymer that changes confirmation rapidly and reversibly in response to an environmental stimulus, for example, temperature, pH, ionic strength, electrical potential, light, magnetic field or ultrasound. See, e.g., Stubbe, et al., Pharmaceutical Res., 21:1732-1740, 2004, which is incorporated herein by reference. Examples of polymers are described in U.S. Pat. Nos. 5,830,207; 6,720,402; and 7,033,571, which are incorporated herein by reference. For example, a hydrogel or other polymer may be used as an environmentally sensitive actuator to control release of the release agent from the reservoir to dissolve the sugar glass composition from an interior of the sugar glass composition to release the vaccine or therapeutic agent from the sugar glass composition in a compartment of the device. An implantable delivery device, oral delivery device, or rectal delivery device may incorporate a hydrogel or other polymer that modulates delivery of the vaccine or therapeutic agent in response to environmental conditions. See, e.g., U.S. Pat. Nos. 6,416,495; 6,571,125; and 6,755,621, which are incorporated herein by reference.

Examples of hydrogels and/or polymers include, but are not limited to, gelled and/or cross-linked water swellable polyolefins, polycarbonates, polyesters, polyamides, polyethers, polyepoxides and polyurethanes such as, for example, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(allyl alcohol). Other suitable polymers include but are not limited to cellulose ethers, methyl cellulose ethers, cellulose and hydroxylated cellulose, methyl cellulose and hydroxylated methyl cellulose, gums such as guar, locust, karaya, xanthan gelatin, and derivatives thereof. For iontophoresis, for example, the polymer or polymers may include an ionizable group such as, for example, (alkyl, aryl or aralkyl) carboxylic, phosphoric, glycolic or sulfonic acids, (alkyl, aryl or aralkyl) quaternary ammonium salts and protonated amines and/or other positively charged species as described in U.S. Pat. Nos. 5,558,633, 6,753,191; 6,589,452; and 6,544,800, which is incorporated herein by reference in its entirety.

Upon removal of the individual thin metal membrane coverings, hydrogel coverings, or polymer coverings on the compartments or reservoirs, and/or disruption or dissolution of reservoirs that include microchannels, nanochannels, microbubbles, microparticles, or nanoparticles embedded in the sugar glass composition, the one or more release agents are delivered from the reservoir into the interior of the sugar glass composition where they act to disrupt or dissolve the sugar glass composition. The one or more release agents can include at least one of water, saline, buffer (e.g., HEPES, Ringer's), biological fluid, physiological fluid (e.g., gastric fluid, saliva, intestinal fluid, blood fluid, interstitial fluid, cerebrospinal fluid, blood fluid, or lymph fluid), oil, or other non-toxic fluid.

Reservoirs that contain one or more release agents can include tubular or particulate microstructures or nanostructures, for example, microchannels, nanochannels, microbubbles, microparticles, or nanoparticles. The tubular or particulate microstructures or nanostructures, as described herein, may be made from a wide variety of materials, for example, organic, inorganic, polymeric, biodegradable, biocompatible and combinations thereof. Non-limiting examples of inorganic materials to make tubular microstructures or nanostructures as described herein include, but are not limited to, iron oxide, silicon oxide, titanium oxide. The inorganic materials may be used in combination with biodegradable monomers and in combination with thin metal membrane coverings, hydrogel coverings, or polymer coverings. Examples of biodegradable monomers formed into tubular or particulate microstructures or nanostructures include polysaccharides, cellulose, chitosan, carboxymethylated cellulose, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides, polyalkyl(cyano)acrylates, poly(trimethylene carbonate) and copolymers, poly(ε-caprolactone) homopolymers and copolymers, polyhydroxybutyrate and polyhydroxyvalerate, poly(ester)urethanes and copolymers, polymethyl-methacrylate and combinations thereof. The carrier may even include or made from polyglutamic or polyaspartic acid derivatives and their copolymers with other amino-acids.

The tubular or particulate microstructures or nanostructures as described herein may be carbon nanochannels, microchannels, nanoparticles, or microparticles that, optionally, in combination with thin metal membrane coverings, hydrogel coverings, or polymer coverings, may contain or channel release agent into an interior of the sugar glass composition. Carbon nanochannels or microchannels are all-carbon hollow graphitic tubes with nanoscale diameter. They can be classified by structure into two main types: single walled carbon nanochannels or microchannels, which consist of a single layer of graphene sheet seamlessly rolled into a cylindrical tube, and multiwalled carbon nanochannels or microchannels, which consist of multiple layers of concentric cylinders. Carbon sources for use in generating carbon nanochannels or microchannels include, but are not limited to, carbon monoxide and hydrocarbons, including aromatic hydrocarbons, e.g., benzene, toluene, xylene, cumene, ethylbenzene, naphthalene, phenanthrene, anthracene or mixtures thereof, non-aromic hydrocarbons, e.g., methane, ethane, propane, ethylene, propylene, acetylene or mixtures thereof; and oxygen-containing hydrocarbons, e.g., formaldehyde, acetaldehyde, acetone, methanol, ethanol or mixtures thereof.

Carbon nanochannels, microchannels, nanoparticles, or microparticles may be synthesized from one or more carbon sources using a variety of methods, e.g., arc-discharge, laser ablation, or chemical vapor deposition (CVD; see, e.g., Bianco, et al., in Nanomaterials for Medical Diagnosis and Therapy. pp. 85-142. Nanotechnologies for the Live Sciences Vol. 10 Edited by Challa S. S. R. Kumar, WILEY-VCH Verlag GmbH & Co. KGaA, Weinheim, 2007, which is incorporated herein by reference).

The arc discharge method can create nanochannels, microchannels, nanoparticles, or microparticles through arc-vaporization of two carbon rods placed end to end, separated by approximately 1 mm, in an enclosure that is filled, for example, with inert gas (e.g., helium, argon) at low pressure (between 50 and 700 mbar). A direct current of 50 to 100 amperes driven by approximately 20 volts creates a high temperature discharge between the two electrodes. The discharge vaporizes one of the carbon rods and forms a small rod-shaped or particle-shaped deposit on the other rod.

Alternatively, carbon nanochannels, microchannels, nanoparticles, or microparticles may be synthesized using laser ablation in which a pulsed or continuous laser energy source is used to vaporize a graphite target in an oven at 1200° C. The oven is filled with an inert gas such as helium or argon, for example, in order to keep the pressure at 500 Ton. A hot vapor plume forms, expands, and cools rapidly. As the vaporized species cool, small carbon molecules and atoms quickly condense to form larger clusters. The catalysts also begin to condense and attach to carbon clusters from which the tubular molecules grow into single-wall carbon nanochannels or microchannels, or nanoparticles or microparticles. The single-walled carbon nanochannels, microchannels, nanoparticles, or microparticles formed in this case are bundled together by van der Waals forces.

Carbon nanochannels, microchannels, nanoparticles, or microparticles may also be synthesized using chemical vapor deposition (CVD). CVD synthesis is achieved by applying energy to a gas phase carbon source such as methane or carbon monoxide, for example. The energy source is used to “crack” the gas molecules into reactive atomic carbon. The atomic carbon diffuses towards a substrate, which is heated and coated with a catalyst, e.g., Ni, Fe or Co where it will bind. The catalyst is generally prepared by sputtering one or more transition metals onto a substrate and then using either chemical etching or thermal annealing to induce catalyst particle nucleation. Thermal annealing results in cluster formation on the substrate, from which the nanochannels, microchannels, nanoparticles, or microparticles will grow. Ammonia may be used as the etchant. The temperatures for the synthesis of nanochannels, microchannels, nanoparticles, or microparticles by CVD are generally within the 650-900° C. range. A number of different CVD techniques for synthesis of carbon nanochannels, microchannels, nanoparticles, or microparticles have been developed, such as plasma enhanced CVD, thermal chemical CVD, alcohol catalytic CVD, vapor phase growth, aero gel-supported CVD and laser-assisted thermal CVD, and high pressure CO disproportionation process (HiPCO). Additional methods describing the synthesis of carbon nanochannels, microchannels, nanoparticles, or microparticles may be found, for example, in U.S. Pat. Nos. 5,227,038; 5,482,601; 6,692,717; 7,354,881 which are incorporated herein by reference.

Carbon nanochannels or microchannels may be synthesized as closed at one or both ends. As such, forming a hollow tube may necessitate cutting the carbon nanochannels or microchannels. Carbon nanochannels or microchannels may be cut into smaller fragments using a variety of methods including but not limited to irradiation with high mass ions, intentional introduction of defects into the carbon nanochannels or microchannels during synthesis, sonication in the presence of liquid or molten hydrocarbon, lithography, oxidative etching with strong oxidating agents, mechanical grinding with diamond balls, or physical cutting with an ultramicrotome (see, e.g., U.S. Pat. No. 7,008,604; Wang et al, Nanotechnol. 18:055301, 2007, which are incorporated herein by reference.) For irradiation with high mass ions, for example, the carbon nanochannels or microchannels are subjected to a fast ion beam, e.g., from a cyclotron, at energies of from about 0.1 to 10 giga-electron volts. Suitable high mass ions include those over about 150 AMU's such as bismuth, gold, uranium and the like. To generate defects that are susceptible to cleavage, the carbon nanochannels or microchannels may be synthesized in the presence of a small amount of boron, for example. For sonication, carbon nanochannels or microchannels may be sonicated in the presence of 1,2-dichloroethane, for example, using a sonicator with sufficient acoustic energy over a period ranging from 10 minutes to 24 hours, for example. For oxidative etching, carbon nanochannels or microchannels may be incubated in a solution containing 3:1 concentrated sulfuric acid:nitric acid for 1 to 2 days at 70° C. For cutting with an ultra-microtome, the carbon nanochannels or microchannels are magnetically aligned, frozen to a temperature of about −60° C., and cut using an ultra-thin cryo-diamond knife.

Once synthesized, carbon nanochannels, microchannels, nanoparticles, or microparticles may be further purified to eliminate contaminating impurities, e.g., amorphous carbon and catalyst particles. Methods for further purification include, but are not limited to, acid oxidation, microfiltration, chromatographic procedures, microwave irradiation, and polymer-assisted purification (see, e.g., U.S. Pat. No. 7,357,906, which is incorporated herein by reference). Chromatography and microfiltration may also be used to isolate a uniformed population of carbon nanochannels, microchannels, nanoparticles, or microparticles with similar size and diameter, for example (see, e.g., Bianco, et al., in Nanomaterials for Medical Diagnosis and Therapy. pp. 85-142. Nanotechnologies for the Live Sciences Vol. Edited by Challa S. S. R. Kumar, WILEY-VCH Verlag GmbH & Co. KGaA, Weinheim, 2007, which is incorporated herein by reference). Alternatively, purified carbon nanochannels, microchannels, nanoparticles, or microparticles may be purchased from a commercial source (from, e.g., Carbon Nanotechologies, Houston, Tex.; Sigma-Aldrich, St. Louis, Mo.).

Alternatively, a tubular or particulate microstructures or nanostructures as described herein may be one or more of peptide microchannels, peptide nanochannels, peptide microbubbles, peptide microparticles, or peptide nanoparticles. Peptide microchannels or peptide nanochannels are extended tubular beta-sheet-like structures and are constructed by the self-assembly of flat, ring-shaped peptide subunits made up of alternating D- and L-amino acid residues as described in U.S. Pat. Nos. 6,613,875 and 7,288,623, and in Hartgerink, et al., J. Am. Chem. Soc. 118:43-50, 1996, which are incorporated herein by reference. For example, gramicidin is a pentadecapeptide which forms a β-helix with a hydrophilic interior and a lipophilic exterior bearing amino acid side chains in membranes and nonpolar solvents. In this instance, the helix length is approximately half of the thickness of a lipid bilayer and as such, two gramicidin molecules form an end-to-end dimer stabilized by hydrogen bonds that spans the lipid bilayer. Peptide nanochannels or microchannels are constructed by highly convergent noncovalent processes by which cyclic peptides rapidly self-assemble and organize into ultra large, well ordered three-dimensional structures, upon an appropriate chemical-induced or medium-induced triggering. The properties of the outer surface and the internal diameter of peptide nanochannels or microchannels may be adjusted by the choice of the amino acid side chain functionalities and the ring size of the peptide subunit employed.

Alternatively, tubular or particulate microstructures or nanostructures as described herein may be a lipid microchannels, lipid nanochannels, lipid microbubbles, lipid microparticles, or lipid nanoparticles. Lipid microstructures or nanostructures are typically formed using self-assembling microtubule-forming diacetylenic lipids, such as complex chiral phosphatidylcholines, and mixtures of these diacetylenic lipids as described in U.S. Pat. Nos. 4,877,501, 4,911,981 and 4,990,291, which are incorporated herein by reference. The synthesis of self-assembling lipid nanochannels or microchannels may be accomplished by combining the appropriate lipids with an alcohol and a water phase which leads to the production of lipid microcylinders by direct crystallization. The formation of the lipid tubules may be modulated by the choice of alcohol and/or combination of alcohols, the ratio of alcohol to water, and variations in the reaction temperature (see, e.g., U.S. Pat. No. 6,013,206, which is incorporated herein by reference). A simple method for generating uniform lipid nanochannels or microchannels from single-chain diacetylene secondary amine salts has been described in Lee, et al., J. Am. Chem. Soc. 126:13400-13405, 2004, which is incorporated herein by reference.

The drug delivery device can include one or more energy transducers that target the compartments including the reservoirs within the sugar glass composition. The energy transducer can target the microchannel, nanochannel, microbubble, microparticle, or nanoparticle reservoirs to release the releasing agent at an interior of the sugar glass composition to dissolve the sugar glass composition at the interior followed by release of the pharmaceutically effective compound in the soluble sugar glass composition from the compartment. The energy transducer can include, but is not limited to, an acoustic energy transducer, magnetic energy transducer, or electrical energy transducer. The energy transducer can include an ultrasonic energy transducer.

In an embodiment the drug delivery device comprises one or more compartments including sugar glass composition that enclose reservoirs for the release agent. The reservoirs for the release agent are connected by conduits in each compartment, and the compartments may be each sealed on the top and bottom with a thin metal membrane covering that may be disrupted by an electric current that heats the metal membrane and causes it to disintegrate. The conduit openings into the compartments may also be covered with a metal membrane. See FIGS. 1 and 2A. Coverings over the compartments and the conduit openings are fabricated using microchip fabrication methods that include sputtering and etching to create metal membranes with 20 nm platinum/300 nm titanium/20 nm platinum and metal traces to supply electricity to the metal membrane coverings on individual compartments of the drug delivery device. See e.g., Maloney et al., J. Controlled Release 109: 244-255, 2005 and U.S. Pat. No. 7,413,846 Ibid., which are incorporated herein by reference. The drug delivery device can include a microchip with circuitry and a small battery to supply current (approximately 0.5 amp) to thermally disrupt individual compartment coverings and conduit openings. A battery and capacitor (with a value of approximately 470 μF) are used to provide current to the metal membrane coverings. For example, a 0.5 amp current may disrupt approximately 72% of the membrane area within approximately 100 μseconds. Individual compartment coverings may be disrupted automatically (i.e., programmed in the microchip) or by external command to execute a drug delivery schedule, meet dosing requirements and deliver multiple medications.

In an embodiment the drug delivery device comprises reservoirs that include nanoparticles or microparticles at least partially embedded in the sugar glass composition. Nanoparticle or microparticle reservoirs containing a release agent such as phosphate buffered saline (PBS) can be prepared from a light-sensitive polymer formulated into nanoparticles that encapsulate the release agent. Nanoparticle or microparticle reservoirs including a light-sensitive polymer can be synthesized using a monomer (4,5 dimethoxy-2-nitrobenzyl alcohol) and adipoyl chloride to yield polymer with molecular weight of 65,000 Daltons (see e.g., Fomina et al., J. Am Chem. Soc. 132: 9540-9542, 2010, which is incorporated herein by reference). The polymer can undergo self-destruction when irradiated with near infrared light at approximately 750 nm wavelength. Alternate polymers sensitive to different wavelengths of light can be used to construct nanoparticle or microparticle reservoirs and coverings for different compartments of the drug delivery device. For example, nanoparticle or microparticle reservoirs produced from a polymer made with 4-bromo7-coumarin self-destruct when irradiated with 740 nm light (see e.g., Fomina et al., Macromolecules 44: 8590-8597, 2011 which is incorporated herein by reference). Laser diodes emitting wavelengths ranging between 404 nm and 785 nm are available from Thorlabs, Newton, N.J. Individual compartments of the drug delivery device corresponding to different therapeutic dosages can be irradiated sequentially or simultaneously to execute a dose and schedule regimen for delivering the pharmaceutically effective compound.

The drug delivery device can include microbubble reservoirs with a specific resonant ultrasound frequency and a specific ultrasound pressure threshold for disruption by cavitation. The microbubbles are produced with varying diameter and lipid shell composition to disrupt by cavitation with varying specific resonant ultrasound frequency and ultrasound pressure threshold. See for example, Dicker et al., Bubble Science, Engineering and Technology 2: 55-64, 2010 and U.S. Patent Appl. No. 2009/0098168 Ibid. which are incorporated herein by reference. Microbubbles with lipid shells containing DSPE-PEG2000 at varying concentrations (e.g., 1, 2.5, 7.5 and 10 mol %) display cavitation pressure thresholds for destruction of 50% of the microbubbles of 0.85, 0.88, 0.93, 1.19 and 1.26 MPa respectively. Also microbubbles with different diameters, e.g., 1.5 μm and 3.0 μIna, have different resonant frequencies, 5.2 MHz and 2.2 MHz respectively. Thus microbubbles encapsulating the release agent, PBS, can be produced with different cavitation pressure thresholds and different resonant frequencies for incorporation with a pharmaceutically effective composition formulated as a glassy substance. The compartments can contain microbubbles with different resonant frequencies and different threshold cavitation pressures which allow exclusive disruption of microbubbles in each compartment by pulsing the compartment at a specific ultrasound frequency and acoustic pressure. An ultrasound transducer combined with an arbitrary waveform generator can be used to pulse the compartment and disrupt the microbubble reservoirs within the sugar glass composition. For example, spherically-focused single-element transducers, 2.25 MHz and 5.0 MHz (available from Panametrics, Inc., Waltham, Mass.) can be used to pulse microbubbles with radii of 3-6 μm at their resonant frequency. An arbitrary waveform generator (e.g., AWG 2021 available from Tektronix, Inc., Beaverton, Oreg. can be used to produce the excitation waveform and a radio frequency amplifier (ENI 3200L available from Bell Electronics NW Inc., Kent, Wash.) can be used to amplify the waveform and energize the transducer (see e.g., U.S. Patent No. 2009/0098168 Ibid.). The disruption of specific microbubbles can be quantified as a function of acoustic pressure, pulse length and frequency.

The drug delivery device comprises one or more compartments including a sugar glass composition that includes at least one of a monosaccharide, disaccharide, or oligosaccharide. The sugar glass composition includes, but is not limited to, at least one of sucrose, glucose, fructose, maltose, mannose, maltulose, iso-maltose, nigerose, cellubiulose, turanose, panose, isomaltotriose, stachyose, nystose, maltotetrose, maltopentose, maltohexose, maltopheptose, ubombo sugar, raffinose, arabinose, galactose, xylose, melibiose, salicin, esculin, arbutin, glycerol, arabinose, adonitol, sorbose, thamnose, dextrose, inulin, dextran, malto-dextran, dulcitol, melezitose, starch, glycogen, gentiobiose, lyxose, tagatose, fucose, arabitol, gluconate, or trehalose. The sugar glass composition includes at least one non-reducing monosaccharide (e.g., methylated version). The sugar glass composition includes at least one of carboxylate, phosphate, nitrate, sulfate, or bisulfate.

The stabilizing glass composition includes, but is not limited to, at least one of monsaccharide glass, disaccharide glass, polysaccharide glass, oligosaccharide glass, trehalose glass, or glucose glass. The stabilizing glass composition can include, but is not limited to, an amino acid, sugar, metal, acrylic, or salt, e.g., an amino acid glass, sugar glass, metal carboxylate glass, borosilicate glass, acrylic glass, aluminum oxynitride glass, Muscovite glass, or calcium phosphate glass. In an embodiment, the glassy substance includes at least one of dextran, phosphatidylcholine, hexuronic acid, or polyethylene glycol. The sugar glass composition includes at least one sugar alcohol. The sugar alcohol includes at least one of trehalose, glucose, sorbitol, mannitol, inositol, erythritol, or lactitol. The sugar glass composition includes at least one of palatnitol, xylitol, glucopyranosyl sorbitol, or glucopyranosyl mannitol.

The sugar glass composition can be spun into hydrophilic fibers for storage or delivery. The fibers can be cut after formation of the solution or mixture, and before or after enclosure in the delivery device.

The one or more pharmaceutically effective compounds can include one or more therapeutic compounds or one or more prophylactic compounds. The therapeutic compounds or the prophylactic compounds can include, but are not limited to, at least one of a vaccine, adjuvant, small molecule (peptide, protein, hormone, nucleic acid, antibody or antibody fragments), biological agent (bacteria, virus, eukaryotic or prokaryotic cell, liposome, phage). The therapeutic compounds or the prophylactic compounds can include, but are not limited to, at least one of an organic or inorganic small molecule, clathrate or caged compound, protocell, coacervate, microcapsule, proteinoid, liposome, vesicle, small unilamellar vesicle, large unilamellar vesicle, large multilamellar vesicle, multivesicular vesicle, lipid layer, lipid bilayer, micelle, organelle, cell, membrane, nucleic acid, peptide, polypeptide, protein, glycopeptide, glycolipid, glycoprotein, sphingolipid, glycosphingolipid, peptidoglycan, lipid, carbohydrate, metalloprotein, proteoglycan, chromosome, nucleus, nitric oxide, nitric oxide synthase, amino acid, micelle, polymer, co-polymer, or piloxymer. Microcapsules can include, but are not limited to, microspheres, microcylinders, or microparticles.

The therapeutic or prophylactic compounds can include, but are not limited to, at least one of an anti-tumor agent, antimicrobial agent, anti-viral agent, analgesic, antiseptic, anesthetic, diagnostic agent, anti-inflammatory agent, vaccine, cell growth inhibitor, cell growth promoter, chemical debridement agent, immunogen, antigen, radioactive agent, apoptotic promoting factor, angiogenic factor, anti-angiogenic factor, hormone, enzymatic factor, enzyme, papain, collagenase, protease, peptidase, elastase, urea, vitamin, mineral, nutraceutical, cytokine, chemokine, probiotic, coagulant, anti-coagulant, phage, prodrug, prebiotic, blood sugar stabilizer, smooth muscle cell activator, epinephrine, adrenaline, neurotoxin, neuro-muscular toxin, Botulinum toxin type A, microbial cell or component thereof, or virus or component thereof. In at least one embodiment, the nutraceutical includes one or more of a flavonoid, antioxidant, beta-carotene, anthocyanin, α-linolenic acid, omega-3 fatty acids, yeast, bacteria, algae, other microorganisms, plant products, or animal products. The analgesic or anesthetic can include, but are not limited to, one or more of any aminoamid or aminoester local anesthetic, ibuprofen, morphine, codeine, aspirin, acetaminophen, lidocaine/lignocaine, ropivacaine, mepivacaine, benzocaine, chloroprocaine, cocaine, cyclomethycaine, dimethocaine/larocaine, propoxycaine, procaine/novocaine, proparacaine, tetracaine/amethocaine, articaine, bupivacaine, carticaine, cinchocaine/dibucaine, etidocaine, levobupivacaine, piperocaine, prilocaine, trimecaine, saxitoxin, or tetrodotoxin.

The therapeutic or prophylactic compounds can include, but are not limited to, at least one anti-inflammatory agent, including but not limited to steroids, non-steroidal anti-inflammatory drugs, topical anti-inflammatory agents, or subcutaneously administered non-steroidal anti-inflammatory drugs (e.g. diclofenac).

The analgesic can include, but is not limited to, but is not limited to one or more of paracetamol (acetaminophen), non-steroidal anti-inflammatory drugs (NSAIDs), salicylates, narcotics, or tramadol. In at least one embodiment, the analgesic includes but is not limited to aspirin, rofecoxib, celecoxib, morphine, codeine, oxycodone, hydrocodone, diamorphine, pethidine, buprenorphine, amitriptyline, carbamazepine, bagapentin, pregabalin, ibuprofen, naproxen, lidocaine, a psychotropic agent, orphenadrine, cyclobenzaprine, scopolamine, atropine, gabapentin, methadone, ketobemidone, fentanyl, or piritramide.

The therapeutic or prophylactic compounds can include, but are not limited to, one or more antiseptic, including one or more of an alcohol, a quaternary ammonium compound, boric acid, hydrogen peroxide, chlorhexidine gluconate, iodine, mercurochrome, octenidine dihydrochloride, phenol (carbolic acid) compounds, sodium chloride, or sodium hypochlorite.

The antiseptic can include, but is not limited to, one or more of povidone-iodine, iodine, ethanol, 1-propanol, 2-propanol/isopropanol, benzalkonium chloride, cetyl trimethylammonium bromide, cetylpyridinium chloride, benzethonium chloride, chlorhexidine, octenidine dihydrochloride, or carbolic acid.

The antimicrobial agent can include, but is not limited to, at least one of an anti-fungal agent, antibiotic agent, anti-bacterial, anti-parasitic agent, or anti-worm agent. In certain instances, the antimicrobial agent may occur in nature, or it may be synthetic.

The therapeutic compounds can include, but are not limited to, one or more anti-tumor agent, at least one of which may also be identified as a cytotoxic agent, or chemotherapy agent. Non-limiting examples of an anti-tumor agent for use as described herein include at least one of an alkylating agent, antimetabolite, anthracycline, plant alkaloid (such as paclitaxel), topoisomerase inhibitor, monoclonal antibody, or tyrosine kinase inhibitor. The therapeutic compounds includes one or more of imatinib, mechlorethamine, cyclophosphamide, chlorambucil, azathioprine, mercaptopurine, vinca alkaloid, taxane, vincristine, vinblastine, vinorelbine, vindesine, podophyllotoxin, etoposide, teniposide, amsacrine, dactinomycin, trastuzumab, cetuximab, rituximab, bevacizumab, dexamethasone, finasteride, tamoxifen, goserelin, telomerase inhibitor, dichloroacetate, aminopterin, methotrexate, pemetrexed, raltitrexed, cladribine, clofarabine, fludarabine, pentostatin, thioguanine, cytarabine, decitabine, fluorouracil/capecitabine, floxuridine, gemcitabine, enocitabine, sapacitabine, chloromethine, cyclophosphamide, ifosfamide, melphalan, bendamustine, trofosfamide, uramustine, carmustine, fotemustine, lomustine, nimustine, prednimustine, ranimustine, semustine, spretpozocin, carboplatin, cisplatin, nedaplatin, oxaliplatin, triplatin tetranitrate, satraplatin, busulfan, mannosulfan, treosulfan, procarbazine, decarbazine, temozolomide, carboquone, ThioTEPA, triaziquone, triethylenemelamine, docetaxel, larotaxel, ortataxel, tesetaxel, vinflunine, ixabepilone, aclarubicin, daunorubicin, doxorubicin, epirubicin, idarubicin, amrubicin, pirarubicin, valrubicin, zorubicin, metoxantrone, pixantrone, actinomycin, bleomycin, mitomycin, plicamycin, hydroxyurea, camptothecin, topotecan, irinotecan, rubitecan, belotecan, altretamine, amsacrine, bexarotene, estramustine, irofulven, trabectedin, cetuximab, panitumumab, trastuzumab, rituximab, tositumomab, alemtuzumab, bevacizumab, edrecolomab, gemtuzumab, axitinib, bosutinib, cediranib, dasatinib, erlotinib, gefitinib, imatinib, lapatinib, lestaurtinib, nilotinib, semaxanib, sorafenib, sunitinib, vandetanib, alvocidib, seliciclib, aflibercept, denileukin diftitox, aminolevulnic acid, efaproxiral, porflmer sodium, talaporfin, temoporfin, verteporfin, alitretinoin, tretinoin, anagrelide, arsenic trioxide, asparaginase/pegaspergase, atrasentan, bortezomib, carmofur, celecoxib, demecolcine, elesclomol, elasamitrucin, etoglucid, lonidamine, lucanthone, masoprocol, mitobronitol, mitoguanzone, mitotane, oblimersen, omacetaxine, sitimagene ceradenovec, tegafur, testolactone, tiazofurine, tipifarnib, or vorinostat.

The therapeutic or prophylactic compounds can include at least one nutraceutical. At least one nutraceutical includes, but is not limited to, one or more of an extract of plant or animal matter (e.g., an oil, aqueous, or solid extract), a vitamin, a mineral, a mixture or solution, a food supplement, a food additive, a food fortification element, or other nutraceutical. At least one nutraceutical includes, but is not limited to, resveratrol, an antioxidant, psyllium, sulforaphane, isoflavonoid, α-linolenic acid, beta-carotene, anthocyanins, phytoestrogens, polyphenols, polyphenons, catechins, benzenediols, tannins, phenylpropanoids, caffeine, alcohol, or others.

The therapeutic or prophylactic compounds include one or more vaccine. The one or more pharmaceutically effective compounds including at least one vaccine includes at least one prophylactic vaccine or therapeutic vaccine. The at least one therapeutic vaccine includes at least one anti-cancer vaccine. The at least one vaccine includes at least one of an anti-tumor agent, antimicrobial agent, anti-viral agent, immunogen, antigen, live microbe, dead microbe, attenuated microbe, microbe or component thereof, live virus, recombinant virus, killed virus, attenuated virus, virus component, plasmid DNA, nucleic acid, amino acid, peptide, protein, glycopeptide, proteoglycan, glycoprotein, glycolipid, sphingolipid, glycosphingolipid, cancer cell or component thereof, organic or inorganic small molecule, or toxoid.

One or more vaccine can include, but not be limited to, vaccines containing killed microorganisms (such as vaccines for flu, cholera, bubonic plague, and hepatitis A), vaccines containing live, attenuated virus or other microorganisms (such as vaccines for yellow fever, measles, rubella, and mumps), live vaccine (such as vaccines for tuberculosis), toxoid (such as vaccines for tetanus, diphtheria, and crotalis atrox), subunit of inactivated or attenuated microorganisms (such as vaccines for HBV, VLP, and HPV), conjugate vaccines (such as vaccines for H. influenzae type B), recombinant vector, DNA vaccination. In at least one embodiment, the at least one vaccine includes but is not limited to rubella, polio, measles, mumps, chickenpox, typhoid, shingles, hepatitis A, hepatitis B, diphtheria, pertussis, rotavirus, influenza, meningococcal disease, pneumonia, tetanus, rattlesnake venom, virus-like particle, or human papillomavirus, or anti-cancer vaccine.

The therapeutic or prophylactic compounds can include, but is not limited to, at least one adjuvant. The at least one adjuvant may include, but not be limited to, one or more organic or inorganic compounds. The at least one adjuvant may include but not be limited to at least one of a liposome, virosome, lipid, phospholipid, mineral salt, single-stranded DNA, double-stranded RNA, lipopolysaccharide, molecular antigen cage, CpG motif, microbial cell wall or component thereof, squalene, oil emulsion, surfactant, saponin, isolated microbial toxin, modified microbial toxin, endogenous immunomodulator, or cytokine.

The one or more pharmaceutically effective compounds stabilized in a sugar glass composition can be produced with multiple layers (e.g., a composition of layers of different therapeutic compounds or the prophylactic compounds and/or different sugar glass compositions). For example, layered sugar glass compositions can include at least two different layers (e.g., including one type of antibody in one layer and another type of antibody in another) to a particular pathogen. The drug delivery device including the layered sugar glass composition is implanted into a subject or administered orally or rectally, and the various layered therapeutic compounds or prophylactic compounds (e.g., antibodies) are released as the layers of glassy substance(s) and the sugar glass composition are disrupted from the interior of the sugar glass composition. Thus, in an embodiment, a layered sugar glass composition allows for extended or time release of at least one therapeutic agent. The reconstitution of the sugar glass composition occurs as a release agent flows through the reservoir, such as a hydrophilic conduit or channel that flows by capillary action or wicking through hydrophilic microfibers. Hydrophilic microfibers can include peptide microchannels, e.g., gramicidin is a pentadecapeptide which forms a β-helix with a hydrophilic interior and a lipophilic exterior bearing amino acid side chains in membranes and nonpolar solvents. In this instance, the helix length is approximately half of the thickness of a lipid bilayer and as such, two gramicidin molecules form an end-to-end dimer stabilized by hydrogen bonds that spans the lipid bilayer. Thus, in an embodiment, no separate reconstitution step is required for administration of the therapeutic compounds or the prophylactic compounds to a subject.

The device, methods, and compositions are further described with reference to the following examples; however, it is to be understood that the methods and compositions are not limited to such examples.

### PROPHETIC EXAMPLES

### Example 1

### Implanted Drug Delivery Device with Multiple Compartments Containing Sugar Glass Formulated Vaccines with Light-Sensitive Nanoparticle Reservoirs and Compartment Coverings

An implantable drug delivery device is constructed with multiple compartments which contain vaccines that target different pathogens and are formulated as glassy sugars that are sequentially delivered from the compartments. To rapidly deliver a vaccine from a compartment, an energy source opens coverings over the compartments and simultaneously opens nanoparticle vesicles/reservoirs that contain a release agent able to dissolve the sugar glass compound from an interior of the sugar glass within the compartment. The dissolved vaccine diffuses outside the compartment into the tissues surrounding the device.

The implantable device is constructed of biocompatible polymer (e.g., polyurethane) in a disc shape (see FIG. 1) with a diameter of approximately 20 mm and a depth of approximately 7.0 mm. The device contains 20 cylindrical compartments that are each approximately 4.0 mm in diameter and 5 mm in depth which hold a volume of approximately 63 μL.

Vaccines targeting different pathogens (e.g., influenza A H1N1, influenza A H3N2, influenza B (Brisbane), Human Immunodeficiency virus (HIV), Hepatitis B virus, meningococci, measles, mumps and rubella) are formulated as glassy substances that include vesicles containing a release agent. See e.g., CDC Vaccine Schedule, which is incorporated herein by reference

Nanoparticle/vesicle reservoir containing a release agent such as phosphate buffered saline (PBS) are prepared from a light-sensitive polymer formulated into nanoparticles that encapsulate the release agent. A light-sensitive polymer is synthesized using a monomer (4,5 dimethoxy-2-nitrobenzyl alcohol) and adipoyl chloride to yield polymer with molecular weight of 65,000 Daltons (see e.g., Fomina et al., J. Am Chem. Soc. 132: 9540-9542, 2010, which is incorporated herein by reference). The polymer undergoes self-destruction when irradiated with near infrared light at approximately 750 nm wavelength. Alternate polymers sensitive to different wavelengths of light are used to construct nanoparticle reservoirs and coverings for different compartments containing different vaccines. For example, a polymer made with 4-bromo7-coumarin self-destructs when irradiated with 740 nm light (see e.g., Fomina et al., Macromolecules 44: 8590-8597, 2011 which is incorporated herein by reference). Nanoparticle reservoirs containing phosphate buffered saline (PBS) (pH 7.4) are prepared from the polymer by emulsification. For example, the light-sensitive polymer, dissolved in 2.5 mL of dichloromethane, is added to 50 mL of PBS (pH 7.4) containing 1% poly(vinyl alcohol) and stirred at 1000 RPM to prepare an emulsion and further emulsification is done with a pressure homogenizer. The nanoparticle reservoirs containing PBS are purified to remove the poly(vinyl alcohol) and are added as a suspension (approximately 2 mg/mL of polymer) to the vaccines, and the mixture is formulated as a sugar glass composition.

Formulations of light-sensitive nanoparticle reservoirs containing a release agent (e.g., PBS (pH 7.4)) and an attenuated viral vaccine are combined with solutions containing sucrose and trehalose and then dessicated to create a sugar glass composition. Methods to stabilize an attenuated virus in a sugar glass composition are described (see e.g., Alcock et al., Sci. Transl. Med. 2: 19ra12, 2010 which is incorporated herein by reference). For example, a modified vaccinia virus Ankara (MVA) that encodes antigens from a pathogen such as the human immunodeficiency virus (HIV) (see e.g., Hanke et al., J. Gen. Virol. 88: 1-12, 2007, which is incorporated herein by reference) is grown on chick embryo fibroblasts and purified to obtain a viral stock. The MVA stock is diluted five-fold in a solution containing 0.25 M sucrose, 0.25 M trehalose, and 2 mg/mL nanoparticles. The mixture is pipetted into the compartments of the implantable device and frozen in liquid nitrogen for 5-10 minutes and freeze-dried. A freeze-dryer (e.g., Heto PowerDry PL6000 available from Thermo Fisher Scientific, Waltham, Mass.) is set to a shelf temperature of −35° C., a condenser temperature of −55° C. and a pressure of 0.220 mbar. After 24 hours the pressure is lowered to 0.060 mbar and the shelf temperature is gradually increased to 20° C. and maintained for 24 hours. The dry vaccine aliquots in the compartments of the device are placed in a vacuum desiccator at room temperature prior to adding polymer coverings to the compartments.

The compartments containing vaccines and light sensitive nanoparticles embedded within the sugar glass composition are coated with the same light-sensitive polymer present in the nanoparticles. For example the light-sensitive copolymer of 4,5 dimethoxy-2-nitrobenzyl alcohol) and adipoyl chloride (see above and Fomina et al., 2010, Ibid.) may be added to the compartments containing the HIV vaccine and the corresponding nanoparticles. The compartments and their contents are frozen in liquid nitrogen, and the polymer (approximately 10 mg/mL in dichloromethane) is added to each well. The dichloromethane is evaporated by applying a vacuum, leaving a light-sensitive polymer covering over each compartment.

The implantable device with multiple compartments containing sugar glass vaccines, nanoparticle reservoirs enclosing release agent, and light-sensitive polymer coverings over the compartments is implanted subcutaneously between the skin and muscle on the upper arm of the subject to be treated. To deliver vaccine from individual compartments a laser tuned to 750 nm, e.g., a Ti:Sapphire laser, Mai Tai HP, available from Spectra Physics, Santa Clara, Calif., is focused on the implanted device to irradiate the compartments containing the HIV vaccine. To deliver different vaccines, the implanted device is irradiated in situ with different wavelengths of light. For example, a laser tuned to 740 nm of light is focused on the implanted device to deliver the compartments covered with polymer containing 4-bromo7-coumarin. Laser diodes emitting wavelengths ranging between 404 nm and 785 nm are available from Thorlabs, Newton, N.J. Individual compartments corresponding to different vaccines can be irradiated sequentially or simultaneously to execute a dose and schedule regimen for immunization.

### Example 2

### Implanted Drug Delivery Device with Multiple Compartments Containing Sugar Glass Formulated Interferon α-2b with Microbubble Reservoirs and Thin Metal Compartment Coverings

An implantable drug delivery device with multiple compartments containing interferon stabilized in a sugar glass composition. The compartments also contain microbubble reservoirs that contain a release agent, phosphate buffered saline (PBS), to dissolve the therapeutic drug formulated as a sugar glass composition. The compartments are protected from physiological fluids by metal membrane coverings. To rapidly deliver interferon, the microbubble reservoirs embedded within the sugar glass composition are disrupted by pulsing with ultrasound waves, dispersing the PBS, which disrupts the sugar glass composition from the interior of the sugar glass composition. The compartment covering is simultaneously opened thus releasing the interferon from the compartment.

The implantable device is constructed of biocompatible polymer (e.g., polyurethane) in a disc shape (see FIG. 1) with a diameter of approximately 20 mm and a depth of approximately 7.0 mm. The device contains 24 cylindrical compartments that are each approximately 4.0 mm in diameter and 5 mm in depth which hold a volume of approximately 65 μL. The compartments are each sealed with a thin metal membrane covering that is disrupted by an electric current that heats the metal membrane and causes it to disintegrate. The coverings are fabricated using microchip fabrication methods that include sputtering and etching to create metal membranes with 20 nm platinum/300 nm titanium/20 nm platinum and metal traces to supply electricity to the metal membrane coverings (see e.g., Maloney et al., J. Controlled Release 109: 244-255, 2005, which is incorporated herein by reference). The implantable device includes a microchip with circuitry and a small battery to supply current (approximately 0.5 amp) to thermally disrupt individual coverings. A battery and capacitor (with a value of approximately 470 μF) are used to provide current to the metal membrane coverings. For example, a 0.5 amp current may disrupt approximately 72% of the membrane area within approximately 100 μseconds.

Microbubble reservoirs that encapsulate a release agent are produced in a microfluidic device and incorporated in the compartments with the sugar glass composition. Microbubbles are prepared using a microfluidic device that produces microbubbles with an inner gas core, a liquid layer containing the release agent and a lipid shell. For example, a microfluidic device constructed from a silicon wafer and polydimethylsilane (PDMS) using microfabrication methods such as soft lithography can be utilized. See e.g., U.S. Patent Appl. No. 2009/0098168 published on Apr. 16, 2009, which is incorporated herein by reference. The device contains a dual flow-focusing region with multiple inlets for gas, liquid layer and lipids and yields microbubbles that are uniformly one diameter, i.e., monodisperse. Perfluorocarbon gas is streamed through liquid sheaths of phosphate buffered saline (PBS) pH 7.4 and a lipid mixture, such as a phospholipid like 1,2distearoyl-sn-glycero-3-phosphocholine or DSPC and a lipopolymer emulsifier such as 1,2-distearoyl-sn-glycero-3-phoshoethanolamine-N-[Poly(ethyleneglycol)2000] or DSPE-PEG2000. Microbubbles with PBS pH 7.4 encapsulated can be disrupted by a pulse of ultrasound waves to release the PBS release agent. Microbubbles with a specific resonant ultrasound frequency and a specific ultrasound pressure threshold for disruption by cavitation are produced by varying the diameter and the lipid shell composition of the microbubbles. See for example, Dicker et al., Bubble Science, Engineering and Technology 2: 55-64, 2010 and U.S. Patent Appl. No. 2009/0098168 Ibid., which are incorporated herein by reference. Microbubbles with lipid shells containing DSPE-PEG2000 at varying concentrations (e.g., 1, 2.5, 7.5 and 10 mol %) display cavitation pressure thresholds for destruction of 50% of the microbubbles of 0.85, 0.88, 0.93, 1.19 and 1.26 MPa respectively. Also microbubbles with different diameters, e.g., 1.5 μm and 3.0 μm, have different resonant frequencies, 5.2 MHz and 2.2 MHz respectively. Thus microbubbles encapsulating the release agent, PBS, are produced with different cavitation pressure thresholds and different resonant frequencies for incorporation with interferon α-2b formulated as a glassy substance.

Pegylated interferon α-2b, an antiviral drug prescribed for Hepatitis C virus infections, is formulated as a sugar glass composition. To produce pegylated interferon α-2b as a sugar glass composition, the pegylated interferon α-2b is formulated as a solution containing trehalose and lyophilized. Methods to stabilize proteins in a glassy substance have been described. See e.g., Amorij et al., Vaccine 25: 6447-6457, 2007, which is incorporated herein by reference. For example, a solution containing approximately 1.5 mg/mL of pegylated interferon α-2b (available from Merck & Co. Inc., Whitehouse Station, N.J.) is supplemented with approximately 1.7% (w/v) trehalose (available from Sigma-Aldrich, St. Louis, Mo.). Microbubbles with a lipid shell encapsulating the release agent, PBS, are added to the mixture, and it is frozen in liquid nitrogen for 5-10 minutes and freeze dried. A freeze-dryer (e.g., Heto PowerDry PL6000 available from Thermo Fisher Scientific, Waltham, Mass.) is set to a shelf temperature of −35° C., a condenser temperature of −55° C. and a pressure of 0.220 mbar. After 24 hours the pressure is lowered to 0.060 mbar, and the shelf temperature is gradually increased to 20° C. and maintained for 24 hours. The dry protein samples with microbubbles encapsulating the release agent, PBS, are transferred to a vacuum desiccator at room temperature.

The compartments of the implanted device are filled with pegylated interferon α-2b formulated as a sugar glass composition and microbubble reservoirs with a lipid shell encapsulating the release agent, PBS. See FIG. 3. The compartments contain microbubbles with different resonant frequencies and different threshold cavitation pressures, which allow exclusive disruption of microbubbles in each compartment by pulsing the compartment at a specific ultrasound frequency and acoustic pressure. An ultrasound transducer combined with an arbitrary waveform generator is used to pulse the compartment and disrupt the microbubble reservoirs within. For example, spherically focused single-element transducers, 2.25 MHz and 5.0 MHz (available from Panametrics, Inc., Waltham, Mass.), are used to pulse microbubbles with radii of 3-6 μm at their resonant frequency. An arbitrary waveform generator (e.g., AWG 2021 available from Tektronix, Inc., Beaverton, Oreg.) is used to produce the excitation waveform, and a radio frequency amplifier (ENI 3200L available from Bell Electronics NW Inc., Kent, Wash.) is used to amplify the waveform and energize the transducer (see e.g., U.S. Patent No. 2009/0098168 Ibid.). The disruption of specific microbubbles is quantified as a function of acoustic pressure, pulse length and frequency.

A patient infected by HCV is prescribed pegylated interferon α-2b to be administered once a week for 24 weeks. The implantable device with 24 compartments containing interferon α-2b and microbubble reservoirs with PBS, pH 7.4 is surgically implanted subcutaneously in the patient's upper arm. The microcircuitry on the device is programmed to disrupt a covering on a single compartment once a week at a specified time, e.g., Mondays at 9 am. Simultaneously, microcircuitry on the implanted device signals wirelessly to a computer controlling the external ultrasound transducer to initiate a program to pulse the compartment with ultrasonic waves at a specific frequency and acoustic pressure to disrupt the microbubbles in the compartment and release PBS into the sugar glass interferon. The compartments are sequentially delivered by disruption of their coverings and release of PBS into the sugar glass composition until completion of the 24 week schedule, at which time the implanted device may signal wirelessly to a computer that the device is ready for removal.

### Example 3

### Implanted Drug Delivery Device with Multiple Compartments Containing Sugar Glass Composition Formulated Insulin with Reservoirs of Releasing Agent Delivered by Conduits

An implantable drug delivery device is constructed from a silicon chip with multiple compartments containing insulin formulated in a sugar glass composition. The compartments have thin metal coverings on the top and bottom to isolate the compartment contents from physiological fluids. The compartments are served by reservoirs, e.g., channels that deliver a release agent, phosphate buffered saline, PBS, to the interior of the compartment and the interior of the sugar glass composition. The channels opening to the compartments are also capped by a thin metal covering at the reservoir. Reservoirs outside the compartments provide PBS to the channels when the metal coverings are disrupted. The device has micro-circuitry, a microcontroller, a micro-battery, a capacitor and RFID coil for wireless communication and power acquisition.

The implantable device is constructed from a silicon wafer using microfabrication methods to create multiple compartments, channels and reservoirs (see FIG. 2A). By using photoresist overlays, etching, and sputtering of metals, multiple compartments with metal coverings are created. See, e.g., U.S. Pat. No. 7,413,846 issued to Maloney et al. on Aug. 19, 2008, which is incorporated herein by reference. The device contains 90 compartments which hold a volume of approximately 65 μL each. Multiple reservoirs for the release agent are connected by conduits leading to each compartment and into the interior of the sugar glass composition, and the compartments are each sealed on the top and bottom with a thin metal membrane covering that is disrupted by an electric current that heats the metal membrane and causes it to disintegrate. The conduit openings from the multiple reservoirs are also covered with a metal membrane. See FIG. 2A. Coverings over the compartments and the conduit openings from the multiple reservoirs are fabricated using microchip fabrication methods that include sputtering and etching to create metal membranes with 20 nm platinum/300 nm titanium/20 nm platinum and metal traces to supply electricity to the metal membrane coverings (see e.g., Maloney et al., J. Controlled Release 109: 244-255, 2005 and U.S. Pat. No. 7,413,846 Ibid., which are incorporated herein by reference). The implantable device includes a microchip with circuitry and a small battery to supply current (approximately 0.5 amp) to thermally disrupt individual compartment coverings and conduit openings. A battery and capacitor (with a value of approximately 470 μF) are used to provide current to the metal membrane coverings. For example, a 0.5 amp current may disrupt approximately 72% of the membrane area within approximately 100 μseconds.

The conduit openings from the multiple reservoirs are covered with a thin metal membrane (see above) prior to filling the multiple reservoirs with releasing agent, e.g., PBS, pH 7.4 by using a microinjector. See e.g., U.S. Pat. No. 8,016,817 B2 issued to Santini, Jr. et al. on Sep. 13, 2011, which is incorporated herein by reference. After forming the compartments including the multiple reservoirs with releasing agent, insulin formulated as a sugar glass composition is loaded into the compartments.

Insulin, a therapeutic protein administered daily to Type I diabetes patients is formulated as a sugar glass composition in a solution containing trehalose and lyophilized. Methods to stabilize proteins in a glassy substance are described. See, e.g., Amorij et al., Vaccine 25: 6447-6457, 2007, which is incorporated herein by reference. For example, a solution containing 100 IU (international units)/mL of human insulin (available from Novo-Nordisk, Bagsvaerd, Denmark) is supplemented with approximately 1.7% (w/v) trehalose (available from Sigma-Aldrich, St. Louis, Mo.), and the mixture is microinjected into the compartments of the device. See e.g., U.S. Pat. No. 8,016,817 Ibid. The loaded device is frozen in liquid nitrogen for 5-10 minutes and freeze dried. A freeze-dryer (e.g., Heto PowerDry PL6000 available from Thermo Fisher Scientific, Waltham, Mass.) is set to a shelf temperature of −35° C., a condenser temperature of −55° C. and a pressure of 0.220 mbar. After 24 hours, the pressure is lowered to 0.060 mbar, and the shelf temperature is gradually increased to 20° C. and maintained for 24 hours. The dry protein samples are transferred to a vacuum desiccator at room temperature and then the insulin, formulated as a sugar glass composition, is loaded into the compartments of the implanted device. Finally, the compartments are covered with a thin metal membrane, and metal tracings are applied to provide current to the coverings.

The implanted device with 90 compartments, each containing approximately 30 IU insulin is programmed to deliver insulin automatically every morning at 7:00 am. The microcontroller on the device delivers approximately 0.5 amp of current to the thin metal coverings over and under a single compartment and to the coverings over the conduit openings to allow flow of release agent, PBS pH 7.4, from the reservoirs to an interior of the sugar glass composition in the compartment (see FIG. 2A). Release agent, PBS pH 7.4, flows from the reservoirs into the compartment, and the insulin sugar glass is rapidly dissolved and flows out of the compartment into the surrounding tissue. The implanted device also has a RFID coil that wirelessly communicates with an external reader to verify the delivery of insulin, the date, the time and the compartment number. Implanted devices with wireless transmission of data and power are described. See e.g., U.S. Pat. No. 7,226,442 B2 issued to Sheppard Jr. et al. on Jun. 5, 2007, which is incorporated herein by reference. The multicompartment device is implanted between the epidermis and muscle of the upper arm using standard surgical methods. The device is removed after approximately 90 days, when the compartments are empty and the external reader indicates all insulin doses are exhausted.

### Example 4

### Implanted Drug Delivery Device with Multiple Compartments Containing a Sugar Glass Formulation of a Vaccine and Compartment Coverings

An implantable drug delivery device is constructed with 10 compartments that contain prophylactic drugs that are formulated in a sugar glass composition. The sugar glass is formed in the compartments with a mold that provides multiple microchannel reservoirs from an exterior to an interior of the sugar glass composition. The compartments have metal membrane coverings that may be opened with a controller to release the drugs.

Prophylactic drugs are formulated in a sugar glass composition and loaded into the compartments of the device prior to adding thin metal membrane coverings. For example, a subunit vaccine is produced as a glassy substance containing trehalose and a hemagglutinin (HA) polypeptide from influenza virus. A sugar glass vaccine is formed by freeze-drying solutions of trehalose. See e.g., Amorij et al., Vaccine 25: 6447-6457, 2007, which is incorporated herein by reference. A sugar glass vaccine solution containing approximately 1.7% (w/v) trehalose (available from Sigma-Aldrich, St. Louis, Mo.) and approximately 360 μg/ml of influenza HA protein (e.g., Influenza Hemagglutinin H1N1 A/California available from Sino Biological Inc., Beijing 100176, P.R. China) is microinjected into the compartments of the implantable device. A mold having a plate with microchannels projecting into an interior of the sugar glass composition is placed on the sugar glass therapeutic composition in the compartment. The sugar glass therapeutic composition is frozen in liquid nitrogen for 5-10 minutes and freeze-dried. A freeze-dryer (e.g., Heto PowerDry PL6000 available from Thermo Fisher Scientific, Waltham, Mass.) is set to a shelf temperature of −35° C., a condenser temperature of −55° C. and a pressure of 0.220 mbar. After 24 hours the pressure is lowered to 0.060 mbar and the shelf temperature is gradually increased to 20° C. and maintained for 24 hours. The dry vaccine aliquots in the compartments of the device are placed in a vacuum desiccator at room temperature. The plate mold is removed by cutting the plate from the embedded microchannels to expose open microchannels to an interior of the sugar glass composition. Thin metal membrane coverings are added to cover the exposed embedded microchannels and the sugar glass composition in the compartments. See FIG. 1 and FIG. 2B.

The implantable drug delivery device is constructed of biocompatible polymer (e.g., polyurethane) in a cylindrical shape with a diameter of approximately 20 mm and a depth of approximately 7.0 mm. See FIG. 1. The device contains 10 cylindrical compartments that are each approximately 6 mm in diameter and 5 mm in depth and hold a volume of approximately 150 μL. The compartments are each sealed with a thin metal membrane covering that is disrupted by an electric current that heats the metal membrane and causes it to disintegrate. The coverings are fabricated using microchip fabrication methods that include sputtering and etching to create metal membranes with 20 nm platinum/300 nm titanium/20 nm platinum and metal tracings to supply electricity to the metal membrane covering. See e.g., Maloney et al., J. Controlled Release 109: 244-255, 2005, which is incorporated herein by reference. The implantable device includes a microchip with circuitry and a small battery to supply current (approximately 0.5 amp) to thermally disrupt individual coverings. A battery and capacitor (with a value of approximately 470 μF) are used to provide current to the metal membrane coverings. For example, a 0.5 amp current may disrupt approximately 72% of the membrane area within approximately 100 microseconds. Individual compartment coverings may be disrupted automatically (i.e., programmed in the microchip) or by external command to execute a drug delivery schedule, meet dosing requirements and deliver multiple medications.

Disruption of the compartment coverings allows physiological fluids (e.g., interstitial fluid, lymph fluid, peritoneal fluid) to enter the compartment and flow through the microchannel reservoirs, dissolving the sugar glass subunit vaccine from the interior of the sugar glass composition in addition to its surface, thereby providing a bolus dosage of the subunit vaccine as it diffuses from the compartment at specifically timed intervals. For example, the influenza subunit vaccine may be delivered from two compartments by disruption of their coverings. Simultaneously an adjuvant formulated as a sugar glass (comprised of cytokines and toll-like receptor ligands) may be delivered from two other compartments. The microchip on the implanted device may communicate wirelessly with a mobile computer (cell phone or laptop) to transmit information on the vaccine and adjuvants that have been delivered, the time and date, and the identification number of the implantable device.

Future vaccinations may be delivered from the implanted device to comply with a vaccination schedule or in response to viral pandemics. A vaccination schedule to provide a primary vaccine and then a “booster” vaccine may be executed automatically by programming the microchip in the implanted device to deliver vaccine doses according to a predetermined schedule. If a viral pandemic arises, the implanted device may be controlled externally via wireless communication to deliver a vaccine dose from the appropriate compartment.

### Example 5

### Oral Drug Delivery Device with One or More Compartments Containing a Sugar Glass Formulation of a Vaccine and Compartment Coverings

An oral drug delivery device is constructed as a cylinder with two compartments that contain prophylactic drugs, live attentuated V. cholerae vaccine, formulated in a sugar glass composition with embedded hydrophilic fibers. The sugar glass is formed in the compartments with a mold that provides multiple hydrophilic fibers that extend from an exterior to an interior of the sugar glass composition. The compartments have metal membrane coverings that may be opened with a controller to release the drugs.

Prophylactic drugs are formulated in a sugar glass composition and loaded into the two compartments of the device prior to adding thin metal membrane coverings. For example, a sugar glass vaccine is formed by freeze-drying solutions of trehalose and a live attentuated vaccine. Methods to stabilize proteins in a glassy substance are described. See, e.g., Amorij et al., Vaccine 25: 6447-6457, 2007 which is incorporated herein by reference. See e.g., U.S. Pat. No. 8,016,817 Ibid. A sugar glass vaccine solution containing approximately 1.7% (w/v) trehalose (available from Sigma-Aldrich, St. Louis, Mo.) and approximately 5×10⁹ lyophilized organisms of a V. cholerae strain is microinjected into the compartments of the oral drug delivery device. See, e.g., Sinclair et al., “Oral vaccines for preventing cholera,” The Cochrane Library, DOI: 10.1002/14651858.CD008603.pub2, published online Mar. 16, 2011, which is incorporated herein by reference. A mold form with hydrophilic fibers projecting from its surface through the length of the cylinder is placed into each compartment prior to microinjecting the treholose/vaccine composition into the compartment. The hydrophilic fibers can be, for example, peptide microchannels. Hydrophilic peptide microchannels are formed from gramicidin, a pentadecapeptide which forms a β-helix with a hydrophilic interior and a lipophilic exterior bearing amino acid side chains in membranes and nonpolar solvents. The hydrophilic gramicidin microchannel has a helix length approximately half of the thickness of a lipid bilayer and as such, two gramicidin molecules form an end-to-end dimer stabilized by hydrogen bonds that spans the lipid bilayer. The loaded device is frozen in liquid nitrogen for 5-10 minutes and freeze dried. A freeze-dryer (e.g., Heto PowerDry PL6000 available from Thermo Fisher Scientific, Waltham, Mass.) is set to a shelf temperature of −35° C., a condenser temperature of −55° C. and a pressure of 0.220 mbar. After 24 hours, the pressure is lowered to 0.060 mbar, and the shelf temperature is gradually increased to 20° C. and maintained for 24 hours. The dry vaccine protein samples in the compartments of the device are transferred to a vacuum desiccator at room temperature. The mold is removed by cutting the fibers, and thin metal membrane coverings with metal tracings are added to cover the exposed fibers and their channels, and the sugar glass composition in the compartments. See FIG. 1 and FIG. 2B. Alternatively, the oral drug delivery device can include the compartment consisting solely of the sugar glass composition/V. cholerae vaccine having embedded hydrophilic fibers.

The oral device is constructed of biocompatible polymer (e.g., polyurethane) in a cylindrical shape with a diameter of approximately 10 mm and a depth of approximately 7.0 mm. See FIG. 1. The device contains up to 5 cylindrical compartments that are each approximately 6 mm in diameter and 5 mm in depth which hold a volume of approximately 150 μL. The compartments are each sealed with a thin metal membrane covering that is disrupted by an electric current that heats the metal membrane and causes it to disintegrate. The coverings are fabricated using microchip fabrication methods that include sputtering and etching to create metal membranes with 20 nm platinum/300 nm titanium/20 nm platinum and metal tracings to supply electricity to the metal membrane covering. See e.g., Maloney et al., J. Controlled Release 109: 244-255, 2005, which is incorporated herein by reference. The oral device includes a microchip with circuitry and a small battery to supply current (approximately 0.5 amp) to thermally disrupt individual coverings. A battery and capacitor (with a value of approximately 470 μF) are used to provide current to the metal membrane coverings. For example, a 0.5 amp current may disrupt approximately 72% of the membrane area within approximately 100 μseconds. Individual compartment coverings may be disrupted automatically (i.e., programmed in the microchip) or by external command to execute a drug delivery schedule, meet dosing requirements and deliver multiple medications.

Alternatively, the oral drug delivery device can include the compartment consisting solely of the sugar glass composition/V. cholerae vaccine having embedded hydrophilic fibers. In this case, the compartment would not have a membrane, metal membrane, or polymer membrane surrounding the sugar glass composition.

Disruption of the compartment coverings allows physiological fluids (e.g., gastric fluid) to enter the compartment, and be conducted by the hydrophilic fiber reservoirs, dissolving the sugar glass subunit vaccine from the interior of the sugar glass composition in addition to its surface, thereby providing a bolus dosage of approximately the vaccine. For example, the vaccine may be delivered from one or more compartments by disruption of their coverings depending on a programmed time of day or a programmed gastric position, such as in the small intestine. The microchip on the oral device may communicate wirelessly with a mobile computer (cell phone or laptop) to transmit information on the vaccine dosage that has been delivered, the time and date, and the identification number of the oral device. Future vaccine dosages may be delivered from the oral device to comply with a dosage schedule.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having ordinary skill in the art will recognize that the subject matter described herein may be implemented in an analog or digital fashion or some combination thereof.

The herein described components (e.g., steps), devices, and objects and the description accompanying them are used as examples for the sake of conceptual clarity and that various configuration modifications using the disclosure provided herein are within the skill of those in the art. Consequently, as used herein, the specific exemplars set forth and the accompanying description are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural or singular terms herein, those having skill in the art can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A drug delivery device comprising:
a device including one or more compartments;
one or more pharmaceutically effective compounds stabilized in a sugar glass composition, at least one of the one or more stabilized pharmaceutically effective compounds in the sugar glass composition enclosed within the one or more compartments; and
one or more reservoirs configured to provide access for one or more release agents to an interior of the sugar glass composition, wherein the one or more reservoirs are configured to controllably dispense the one or more release agents to disrupt the sugar glass composition from the interior of the sugar glass composition.

2. The device of claim 1, comprising:
a controller configured to activate the one or more reservoirs to controllably dispense the one or more release agents to disrupt the sugar glass composition and to initiate release of the one or more therapeutic compounds from the sugar glass composition.

3. The device of claim 2, comprising an energy transducer configured to degrade one or more membranes or covers on the one or more reservoirs to initiate release of the one or more release agents into the interior of the sugar glass composition.

4. The device of claim 1, wherein the drug delivery device is an implantable delivery device.

5. The device of claim 1, wherein the drug delivery device is an orally deliverable or rectally deliverable device.

6. The device of claim 1, wherein the one or more reservoirs are at least partially embedded in the sugar glass composition.

7. The device of claim 6, wherein the one or more reservoirs comprise a channel to the interior of the sugar glass composition.

8. The device of claim 1, wherein the one or more reservoirs are completely embedded within the sugar glass composition.

9. The device of claim 1, wherein the one or more reservoirs comprise one or more conduits or channels to provide access to the release agent within one or more containment vessels.

10. The device of claim 1, wherein the one or more reservoirs comprise one or more receptacles for the release agent.

11. The device of claim 1, wherein the one or more reservoirs comprise one or more conduits or channels to provide access to physiological fluids outside the drug delivery device.

12. (canceled)

13. The device of claim 1, wherein the one or more release agents are configured to disrupt the sugar glass composition from the interior to an exterior of the sugar glass composition.

14. The device of claim 1, wherein the one or more reservoirs comprise one or more physical channels having a dispensing end proximal to the interior of the sugar glass composition, wherein the one or more release agents are configured to pass through the one or more physical channels and through the dispensing end to disrupt the sugar glass composition at the interior of the sugar glass composition.

15. The device of claim 1, wherein the one or more reservoirs comprise one or more conductive components having a dispensing end proximal to the interior of the sugar glass composition, wherein the one or more release agents are configured to pass through the one or more conductive components and controllably dispense through the dispensing end to disrupt the sugar glass composition at the interior of the sugar glass composition.

16. The device of claim 15, wherein the one or more conductive components comprise one or more hydrophilic fibers configured to initiate hydration by controllably dispensing the one or more release agents through the dispensing end to the interior of the sugar glass composition.

17. The device of claim 15, wherein the one or more conductive components comprise one or more microchannels or nanochannels configured to initiate hydration by controllably dispensing the one or more release agents through the dispensing end to the interior of the sugar glass composition.

18. The device of claim 1, wherein the one or more reservoirs comprise one or more microparticles or microvesicles configured to initiate hydration by controllably dispensing the one or more release agents through the dispensing end to the interior of the sugar glass composition.

19. The device of claim 6 comprising,
one or more contaimnent vessels distal to the interior of the sugar glass composition, the one or more containment vessels configured to contain the one or more release agents and configured to deliver the one or more release agents through the one or more at least partially embedded reservoirs to the interior of the sugar glass composition.

20. The device of claim 6 comprising,
one or more contaimnent vessels at the interior of the sugar glass composition, the one or more containment vessels configured to contain the one or more release agents and configured to deliver the one or more release agents through the one or more at least partially embedded reservoirs toward a region proximal to the interior of the sugar glass composition.

21. The device of claim 1, wherein the one or more release agents comprise an aqueous solution, a physiologic solution, an ionic solution, a non-physiologic pH solution, an enzymatic agent, a degradative agent, or a biochemical agent.

22. The device of claim 2, wherein the controller is configured to activate the one or more reservoirs to controllably dispense the one or more release agents in response to one or more exogenous components.

23. The device of claim 22, wherein the one or more exogenous components comprises a biochemical agent indicative of an environmental condition.

24. The device of claim 22, wherein the one or more exogenous components comprises a pathogenic agent or an environmental agent.

25. The device of claim 22, wherein the one or more reservoirs comprise one or more encapsulation matrices embedded in the sugar glass composition.

26. The device of claim 22, wherein the one or more reservoirs comprise one or more controlled release polymers embedded in the sugar glass composition.

27. The device of claim 22, wherein the one or more reservoirs comprise one or more covers configured to be activated by the one or more controllers.

28. The device of claim 2, wherein the controller is configured to activate the one or more reservoirs to controllably dispense the one or more release agents in response to one or more endogenous components.

29. The device of claim 28, wherein the one or more endogenous components are indicative of a disease or condition in a vertebrate subject.

30. The device of claim 28, wherein the one or more endogenous components comprise physiologic fluid, physiologic pH, physiologic analytes, or biomarkers in a vertebrate subject.

31. The device of claim 28, wherein the one or more endogenous components comprise a biochemical agent present in the vertebrate subject and indicative of a disease or condition in a vertebrate subject.

32. The device of claim 28, wherein the one or more reservoirs comprise one or more controlled release polymers.

33. The device of claim 32, wherein the one or more controlled release polymers comprise one or more hydrogels.

34. The device of claim 28, wherein the one or more reservoirs comprise one or more covers configured to be activated by the one or more controllers.

35. The device of claim 1, wherein the one or more release agents comprise one or more endogenous components present in a vertebrate subject to disrupt the sugar glass composition from the interior of the sugar glass composition.

36. The device of claim 35, wherein the one or more endogenous components comprise one or more physiological fluids.

37. The device of claim 35, wherein the one or more reservoirs comprise one or more encapsulation matrices including one or more encapsulated release agents.

38. The device of claim 37, wherein the one or more encapsulation matrices comprise pressurized microcapsules in the sugar glass composition.

39. The device of claim 38, wherein the pressurized microcapsules are configured to release the one or more release agents from the pressurized microcapsules into the sugar glass composition in a time dependent manner.

40. The device of claim 38, wherein the pressurized microcapsules are configured to release the one or more release agents into the sugar glass composition responsive to acoustic energy.

41. The device of claim 39, comprising an energy transducer configured to initiate release of the one or more encapsulated release agents from the pressurized microcapsules into the sugar glass composition in the time dependent manner.

42. The device of claim 37, wherein the one or more encapsulation matrices comprise one or more tuned microcapsules in the sugar glass composition.

43. The device of claim 42, wherein the one or more tuned microcapsules are responsive to two or more different tunings.

44. The device of claim 42, comprising an energy transducer configured to initiate release of the one or more encapsulated release agents from the one or more tuned microcapsules into the sugar glass composition.

45. The device of claim 44, wherein the one or more tuned microcapsules are configured to release the one or more release agents into the sugar glass composition responsive to acoustic energy.

46. The device of claim 44, wherein the energy transducer is configured to initiate release of the one or more encapsulated release agents from the one or more tuned microcapsules into the sugar glass composition in a time dependent manner.

47. The device of claim 44, wherein the energy transducer is an ultrasonic energy transducer.

48. The device of claim 3, wherein the energy transducer comprises an acoustic energy transducer, ultrasonic energy transducer, magnetic energy transducer, or electrical energy transducer.

49. The device of claim 3, wherein the energy transducer is configured to be internal or external to the device.

50. The device of claim 1, wherein the one or more reservoirs are configured to be activated to release the one or more release agents by at least one of pressure variation, temperature variation, or variation in wavelength exposure to radiation.

51. The device of claim 1, wherein the pharmaceutically effective compound comprises a therapeutic compound or a prophylactic compound.

52. The device of claim 1, wherein the pharmaceutically effective compound comprises at least one of a vaccine, an adjuvant, a small molecule, or a biological agent.

53. The device of claim 1, wherein the sugar glass composition comprises at least one of a monosaccharide, a disaccharide, a polysaccharide, or an oligosaccharide.

54. The device of claim 1, wherein the sugar glass composition comprises at least one of trehalose glass, glucose glass, or sugar glass.

55. The device of claim 1, wherein the sugar glass composition comprises at least one of dextran, phosphatidylcholine, hexuronic acid, polyethylene glycol, or sugar alcohol.

56-171. (canceled)
